(19) United States
(12) Patent Application Publication
(10) Pub. No.: US 2023/0348873 A1
(43) Pub. Date: Nov. 2, 2023

(54) NUCLEASE-MEDIATED NUCLEIC ACID MODIFICATION

(71) Applicant: The Regents of the University of Michigan, Ann Arbor, MI (US)

(72) Inventors: YUQING CHEN, Ann Arbor, MI (US); JIE XU, Ann Arbor, MI (US); JIFENG ZHANG, Ann Arbor, MI (US); DONGSHAN YANG, Ann Arbor, MI (US); LINYUAN MA, Ann Arbor, MI (US)

(21) Appl. No.: 18/021,729

(22) PCT Filed: Aug. 17, 2021
(86) PCT No.: PCT/US2021/046247

§ 371 Date: Feb. 16, 2023

Related U.S. Application Data
(60) Provisional application No. 63/067,379, filed on Aug. 19, 2020.

(51) Int. Cl. C12N 9/22; C07K 14/415; C12N 15/10

(52) U.S. Cl. CPC C12N 9/22; C07K 14/415; C12N 15/102; C12N 2310/20

(57) ABSTRACT

Provided herein is technology relating to molecular biological manipulation of genes and genomes and particularly, but not exclusively, to CRISPR (Clustered Regularly Interspaced Short Palindromic Repeats) methods, compositions, systems, and kits for improved genetic editing.

This application claims priority to U.S. provisional patent application Ser. No. 63/067,379, filed Aug. 19, 2020, which is incorporated herein by reference in its entirety.

### FIELD

Provided herein is technology relating to molecular biological manipulation of genes and genomes and particularly, but not exclusively, to CRISPR (Clustered Regularly Interspaced Short Palindromic Repeats) methods, compositions, systems, and kits for improved genetic editing.

### BACKGROUND

CRISPR/Cas9 (CRISPR associated protein 9) is widely used for gene editing. However, CRISPR and related technologies used for gene editing has a low efficiency of “knocking in” (KI) large fragment sequences (e.g., a reporter gene) at target sites. In particular, the efficiency of knock in is often below 1%. Accordingly, improved technologies are needed.

### SUMMARY

Provided herein is a technology related to a modified CRISPR/Cas9 for improved integration of knock-in nucleic acid inserts at target sites. In some embodiments, the technology increases by several fold the KI efficiency of large size inserts at a range of loci in human cells (e.g., primary cells, pluripotent stem cells, and adult stem cells). Furthermore, the CRISPR technology provided herein significantly reduces off-target integration relative to conventional CRISPR approaches, e.g., using a conventional Cas9 protein. As described herein, the improved CRISPR technology finds use in broad applications related to gene editing research and therapeutics.

Related technologies have fused certain peptides to genome editing nucleases (GEN) to improve the efficacy and safety of genome editing using the protein fusion comprising the GEN and peptide. In particular, some previous technologies have fused exon 27 from the BRCA2 gene (also known as “BRCA2 exon 27” or “BE27”), a peptide of thirty-six amino acids (AA), to Streptococcus pyogenes Cas9 (“spCas9”). See, e.g., Int'l pat. app. No. PCT/US2019/030913, incorporated herein by reference.

The technology provided herein provides new GEN protein fusions that have been specifically modified by introducing substitutions of amino acids in the BE27 polypeptide that are involved in homology-directed repair processes. During the development of embodiments of the technology provided herein, individual amino acids of BE27 were identified that are important for homology directed repair (HDR) processes. In particular, data collected during these experiments indicated that the serine at amino acid position 15 (S15) and/or the serine at amino acid position 22 (S22) are particularly involved in HDR. In subsequent experiments, new Cas9 variants (“miCas9-A”, “miCas9-Y”, and “miCas9-D”) were constructed by fusing spCas9 with BE27 variants that comprise amino acid substitutions at S15 and/or S22. In particular, Cas9 variants were constructed by fusing spCas9 to a BE27 polypeptide having a substitution of alanine for the serine at amino acid 15 (S15A), a substitution of tyrosine for the serine at amino acid 22 (S22Y), or a substitution of aspartic acid for the serine at amino acid 22 (S22D). The BE27 polypeptide variants are named BE27-S15A, BE27-S22Y, and BE27-S22D, respectively, and were fused to spCas9 to produce the new Cas9 fusion polypeptide variants named miCas9-A, miCas9-Y, and miCas9-D, respectively (collectively, “miCas9-A/Y/D” or “miCas9 variants”).

During the development of embodiments of the technology described herein, the miCas9-A/Y/D variants produced significantly reduced on-target and off-target insertion and deletion (indel) events without compromising precise gene editing efficiencies. Further, experiments indicated that the miCas9-A/Y/D variants also produced increased rates of large-sized gene knock-ins. Accordingly, each of the BE27 variant peptides (BE27-S15A, BE27-S22Y, or BE27-S22D) provides a universal motif that can be fused with any GEN that produces double-stranded breaks in nucleic acid to improve modification of nucleic acids (e.g., gene editing), e.g., to produce a GEN-BE27-A/Y/D fusion protein having improved efficacy and safety profiles in genome editing applications.

Accordingly, provided herein is technology related to a GEN-BE27 variant fusion protein comprising a gene editing nuclease domain and a BE27 variant domain. In some embodiments, the gene editing nuclease domain comprises a CRISPR-associated system protein, a portion thereof, a homolog thereof, or a modified version thereof. In some embodiments, the gene editing nuclease domain comprises a Cas protein, a portion thereof, a homolog thereof, or a modified version thereof (e.g., a Cas9 (e.g., a spCas9)). In some embodiments, the Cas protein selected is Cas1, Cas1B, Cas2, Cas3, Cas4, Cas5, Cas6, Cas7, Cas8, Cas9, Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, or xCas9; a portion thereof a homolog thereof or a modified version thereof. In some embodiments, the gene editing nuclease domain comprises a TALEN, a portion thereof, a homolog thereof, or a modified version thereof. In some embodiments, the gene editing nuclease domain comprises ZFN, a portion thereof, a homolog thereof, or a modified version thereof.

In some embodiments, the GEN-BE27 variant fusion comprises one BE27 variant domain. In some embodiments, the GEN-BE27 variant fusion comprises a plurality of BE27 variant domains (e.g., 2, 3, 4, 5, 6, 7, 8, 9, 10, 11, 12, 13, 14, 15, 16, 17, 18, 19, 20, or more than 20 BE27 variant domains). In some embodiments, the GEN-BE27 variant fusion comprises a plurality of BE27 variant domains (e.g., 2, 3, 4, 5, 6, 7, 8, 9, 10, 11, 12, 13, 14, 15, 16, 17, 18, 19, 20, or more than 20 BE27 variant domains), wherein each BE27 variant domain is independently selected from BE27-S15A, BE27-S22Y, and/or BE27-S22D. In some embodiments, the GEN-BE27 variant fusion comprises 1-10 BE27 variant domains (e.g., 1, 2, 3, 4, 5, 6, 7, 8, 9, or 10 BE27 variant domains). In some embodiments, the GEN-BE27 variant fusion comprises 1-10 BE27 variant domains (e.g., 1, 2, 3, 4, 5, 6, 7, 8, 9, or 10 BE27 variant domains) and each BE27 variant domain is independently selected from BE27-S15A, BE27-S22Y, and/or BE27-S22D. In some embodiments, the BE27 variant domain comprises an S15A substitution. In some embodiments, the BE27 variant domain comprises an S22Y substitution. In some embodiments, the BE27 variant domain comprises an S22D substitution.

Furthermore, in some embodiments, the technology provides a composition comprising a GEN-BE27 variant fusion protein as described herein. In some embodiments, the composition further comprises a gRNA. In some embodiments, the composition further comprises a donor nucleic acid. In some embodiments, the composition further comprises a target nucleic acid. In some embodiments, the composition comprises a donor nucleic acid comprising 100 to 1000 bp (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 bp). In some embodiments, the composition comprises a donor nucleic acid comprising 1000 to 10,000 bp (e.g., 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 bp). In some embodiments, compositions further comprise a RAD51 protein or a nucleic acid encoding a RAD51 protein. In some embodiments, compositions further comprise a plurality of RAD51 proteins. In some embodiments, compositions further comprise a nucleic acid comprising a knockin. In some embodiments, the nucleic acid comprising a knockin comprises a sequence of the donor nucleic acid.

In some embodiments, the technology provides methods. For example, in some embodiments, the technology provides methods of producing a knockin in a target nucleic acid. In some embodiments, methods comprise contacting a target nucleic acid with a GEN-BE27 variant fusion protein. In some embodiments, methods comprise contacting a target nucleic acid with a ribonucleoprotein comprising a GEN-BE27 variant fusion protein and a gRNA comprising a sequence complementary to the target nucleic acid. In some embodiments, methods comprise providing a donor nucleic acid comprising a knockin sequence. In some embodiments, methods comprise introducing the ribonucleoprotein into a cell. In some embodiments, methods comprise introducing said ribonucleoprotein and said donor nucleic acid into a cell. In some embodiments of methods, the GEN-BE27 variant fusion protein comprises a BE27 variant domain comprising an S15A substitution. In some embodiments of methods, the GEN-BE27 variant fusion protein comprises a BE27 variant domain comprising an S22Y substitution. In some embodiments of methods, the GEN-BE27 variant fusion protein comprises a BE27 variant domain comprising an S22D substitution. In some embodiments of methods, the GEN-BE27 variant fusion protein comprises a gene editing nuclease domain that is a CRISPR-associated system protein, a portion thereof, a homolog thereof, or a modified version thereof. In some embodiments of methods, the GEN-BE27 variant fusion protein comprises a gene editing nuclease domain that is a Cas protein (e.g., a Cas9 (e.g., a spCas9)), a portion thereof, a homolog thereof, or a modified version thereof. In some embodiments of methods, the GEN-BE27 variant fusion protein comprises a gene editing nuclease domain that is a Cas protein selected from the group consisting of Cas1, Cas1B, Cas2, Cas3, Cas4, Cas5, Cas6, Cas7, Cas8, Cas9, Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, and xCas9; a portion thereof a homolog thereof or a modified version thereof. In some embodiments of methods, the GEN-BE27 variant fusion protein comprises a gene editing nuclease domain that is a TALEN, a portion thereof, a homolog thereof, or a modified version thereof. In some embodiments of methods, the GEN-BE27 variant fusion protein comprises a gene editing nuclease domain that is a ZFN, a portion thereof, a homolog thereof, or a modified version thereof.

Further embodiments relate to kits. In some embodiments, kits comprise a GEN-BE27 variant fusion protein as described herein (e.g., a GEN-BE27 variant fusion comprising BE27-S15A, BE27-S22Y, and/or BE27-S22D).

Further embodiments relate to systems. In some embodiments, systems comprise a GEN-BE27 variant fusion protein as described herein (e.g., a GEN-BE27 variant fusion comprising BE27-S15A, BE27-S22Y, and/or BE27-S22D).

Further embodiments provide uses of a GEN-BE27 variant fusion protein (e.g., a GEN-BE27 variant fusion comprising BE27-S15A, BE27-S22Y, and/or BE27-S22D) to produce a transgenic cell. In some embodiments, the technology provides use of a GEN-BE27 variant fusion protein (e.g., a GEN-BE27 variant fusion comprising BE27-S15A, BE27-S22Y, and/or BE27-S22D) to produce a transgenic animal.

In some embodiments, the technology provides a nucleic acid encoding a GEN-BE27 variant fusion protein (e.g., a GEN-BE27 variant fusion comprising BE27-S15A, BE27-S22Y, and/or BE27-S22D). In some embodiments, the technology provides a vector comprising a nucleic acid encoding a GEN-BE27 variant fusion protein (e.g., a GEN-BE27 variant fusion comprising BE27-S15A, BE27-S22Y, and/or BE27-S22D). In some embodiments, the technology provides a cell comprising a GEN-BE27 variant fusion protein (e.g., a GEN-BE27 variant fusion comprising BE27-S15A, BE27-S22Y, and/or BE27-S22D). In some embodiments, the technology provides a cell comprising a nucleic acid encoding a GEN-BE27 variant fusion protein (e.g., a GEN-BE27 variant fusion comprising BE27-S15A, BE27-S22Y, and/or BE27-S22D). In some embodiments, the technology provides a cell expressing a GEN-BE27 variant fusion protein (e.g., a GEN-BE27 variant fusion comprising BE27-S15A, BE27-S22Y, and/or BE27-S22D). In some embodiments, the technology a cell expressing a nucleic acid encoding a GEN-BE27 variant fusion protein (e.g., a GEN-BE27 variant fusion comprising BE27-S15A, BE27-S22Y, and/or BE27-S22D). In some embodiments, a transgenic animal expressing a GEN-BE27 variant fusion protein (e.g., a GEN-BE27 variant fusion comprising BE27-S15A, BE27-S22Y, and/or BE27-S22D). In some embodiments, the technology provides a transgenic animal expressing a nucleic acid encoding a GEN-BE27 variant fusion protein (e.g., a GEN-BE27 variant fusion comprising BE27-S15A, BE27-S22Y, and/or BE27-S22D).

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a photograph of an agarose gel after electrophoresis of products from a T7 endonuclease I assay (T7EI assay; see, e.g., Guschin et al. (2010) “A rapid and general assay for monitoring endogenous gene modification” Methods Mol Biol 649: 247-56, incorporated herein by reference) used to evaluate off-target indel events at EMX1. The experimental results indicated that miCas9-Y, miCas9-A, and miCas9-D produced decreased or undetectable off-target indel events at EMX1 relative to wild-type Streptococcus pyogenes Cas9 (spCas9) and a fusion of Cas9 with wild-type BE27 (miCas9), which both produced detectable off-target indel events at EMX1. A fusion of spCas9 with a BE27 comprising a substitution of glutamic acid for the serine at amino acid 22 (miCas9-S22E) was detected to produce off-target indel events at EMX1.

FIG. 2 is a photograph of an agarose gel after electrophoresis of products from a T7EI assay used to evaluate off-target indel events at FANCF2. The experimental results indicated that miCas9-Y, miCas9-A, and miCas9-D produced decreased or undetectable off-target indel events at EMX1 relative to wild-type Streptococcus pyogenes Cas9 (spCas9) and a fusion of Cas9 with wild-type BE27 (miCas9), which both produced detectable off-target indel events at FANCF2.

FIG. 3 is a photograph of an agarose gel after electrophoresis of products from a T7EI assay used to evaluate off-target indel events at VEGFA3. The experimental results indicated that miCas9-Y, miCas9-A, and miCas9-D produced decreased or undetectable off-target indel events at VEGFA3 relative to wild-type Streptococcus pyogenes Cas9 (spCas9) and a fusion of Cas9 with wild-type BE27 (miCas9), which both produced detectable off-target indel events at VEGF3.

FIG. 4 is a bar plot showing the increased relative efficiencies of miCas9-Y, miCas9-A, miCas9-D, and miCas9-S22E for producing large-size knock-ins at an AAVS1 target site relative to wild-type Streptococcus pyogenes Cas9 (spCas9). miCas9-A also was detected to have an increased efficiency of producing large-size knock-ins at the AAVS1 target site relative to wild-type Streptococcus pyogenes Cas9 (spCas9) and a fusion of Cas9 with wild-type BE27 (miCas9).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is a technology related to a modified CRISPR/Cas9 for improved integration of knock-in nucleic acid inserts at target sites. In some embodiments, the technology the KI efficiency of large size inserts at a range of loci in human cells (e.g., primary cells, pluripotent stem cells, and adult stem cells). Furthermore, the CRISPR technology provided herein significantly reduces off-target integration relative to conventional CRISPR approaches, e.g., using a conventional Cas9 protein. As described herein, the improved CRISPR technology finds use in broad applications related to gene editing research and therapeutics.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “sequencing-free” method does not comprise a sequencing step, etc.

As used herein, the term “gene editing nuclease-BE27 variant fusion” or “GEN-BE27 variant fusion” refers to a polypeptide comprising: 1) a gene editing nuclease domain (e.g., a CRISPR-associated system protein (Cas protein (e.g., a Cas9 protein (e.g., spCas9) or similar as described herein)), a transcription activator-like effector nuclease (TALEN), a zinc-finger nuclease (ZFN), a meganuclease, or variants or modified versions thereof); and 2) a domain comprising one or more amino acid sequences comprising a modified exon 27 from BRCA2 (e.g., a “BE27 variant” (e.g., exon 27 from BRCA2 comprising one or more amino acid substitutions, e.g., at serine 15 and/or serine 22); see discussion below). Accordingly, in some embodiments, the GEN-BE27 variant fusion is a Cas9 variant comprising Cas9 fused to a BE27 polypeptide comprising one or more amino acid substitutions, e.g., a spCas9 polypeptide fused (e.g., at its C-terminus) to a BE27 polypeptide having a substitution of alanine for the serine at amino acid 15 (S15A), a substitution of tyrosine for the serine at amino acid 22 (S22Y), or a substitution of aspartic acid for the serine at amino acid 22 (S22D) (e.g., BE27-S15A, BE27-S22Y, or BE27-S22D) to produce a Cas9 variant, e.g., a miCas9-A, miCas9-Y, or miCas9-D). That is, in some embodiments, the GEN-BE27 variant fusion is a Cas9 variant, referred to herein as a miCas9-A, miCas9-Y, or miCas9-D, comprising an N-terminal domain that is spCas9 and a C-terminal domain comprising BE27-S15A, BE27-S22Y, or BE27-S22D, respectively.

As used herein, the term “BE27” refers to the nucleotide sequence of exon 27 from the BRCA2 gene or a polypeptide encoded by exon 27 from the BRCA2 gene.

As used herein, the term “BE27 variant” refers the nucleotide sequence of exon 27 from the BRCA2 gene comprising one or more mutations or a polypeptide encoded by exon 27 from the BRCA2 gene comprising one or more mutations, e.g., a polypeptide comprising one or more amino acid substitutions. In some embodiments, a BE27 variant is a BE27 nucleotide sequence encoding a BE27 polypeptide or a BE27 polypeptide having a substitution of an amino for the serine at amino acid 15 (S15) or a substitution of an amino acid for the serine at amino acid 22 (S22). In some embodiments, a BE27 variant is a BE27 nucleotide sequence encoding a BE27 polypeptide or a BE27 polypeptide having a substitution of alanine for the serine at amino acid 15 (S15A), a substitution of tyrosine for the serine at amino acid 22 (S22Y), or a substitution of aspartic acid for the serine at amino acid 22 (S22D) (e.g., referred to herein as “BE27-S15A”, “BE27-S22Y”, and “BE27-S22D”, respectively).

In some embodiments, a GEN-BE27 variant fusion comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more BE27 variant domains. In some embodiments, a ribonucleoprotein as described herein comprises a polypeptide that is a GEN-BE27 variant fusion as described herein and a gRNA as described herein. In some embodiments, the GEN-BE27 variant fusion comprises a gene editing nuclease that is a Cas9 or a protein having an activity similar to a Cas9 (e.g., a Cpf1 or other Cas9-like protein or Cas9 homolog as described herein) fused to one or more BE27 variant domains (e.g., 1, 2, 3, 4, 5, 6, 7, 8, 9, 10, 11, 12, 13, 14, 15, 16, 17, 18, 19, 20, 21, 22, 23, 24, or 25 BE27 variant domains).

As used herein, the term “miCas9” refers to a nucleotide sequence encoding a polypeptide comprising Cas9 fused to a BE27 variant or a polypeptide comprising Cas9 fused to a BE27 variant. As used herein, the terms “miCas9-A”, “miCas9-Y”, and “miCas9-D” refer to a nucleotide sequence encoding a polypeptide comprising Cas9 (e.g., spCas9) fused to BE27-S15A, BE27-S22Y, and BE27-S22D, respectively, or a polypeptide comprising Cas9 (e.g., spCas9) fused to BE27-S15A, BE27-S22Y, and BE27-S22D, respectively. In some embodiments, a miCas9 comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more BE27 variant domains. The technology is not limited to a miCas9 comprising a Cas9, but includes any genome editing nuclease (e.g., any CRISPR protein and/or protein having CRISPR activity or activity similar to Cas9).

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982), incorporated herein by reference). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 2002, 41(14), 4503-4510, incorporated herein by reference) and U.S. Pat. No. 5,034,506, incorporated herein by reference), locked nucleic acid (LNA; see Wahlestedt et al., Proc. Natl. Acad. Sci. U.S.A., 2000, 97, 5633-5638, incorporated herein by reference), cyclohexenyl nucleic acids (see Wang, J. Am. Chem. Soc., 2000, 122, 8595-8602, incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

Furthermore, the terms “nucleic acid”, “polynucleotide”, “nucleic acid sequence”, “nucleotide sequence”, and “oligonucleotide” are used interchangeably. They refer to a polymeric form of nucleotides of any length, either deoxyribonucleotides or ribonucleotides, or analogs thereof. Polynucleotides may have any three dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996, each of which is incorporated herein by reference. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Nucleotides in DNA are indicated herein using single letter codes as follows: A (adenine), T (thymine), C (cytosine), and G (guanine); nucleotides in RNA are indicated using single letter codes as follows: A (adenine), U (uracil), G (guanine), and C (cytosine). These nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. As used herein, standard codes for degenerate bases in DNA or RNA are used as follows: R (G or A), Y (T/U or C), M (A or C), K (G or T/U), S (G or C), W (A or T/U), B (G or C or T/U), D (A or G or T/U), H (A or C or T/U), V (A or G or C), or N (A or G or C or T/U), gap (−).

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner, herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152, each of which is incorporated herein by reference). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the term “% sequence identity” refers to the percentage of nucleotides or nucleotide analogs in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to a difference or multiple differences in nucleic acid sequence between two nucleic acids. For example, a wild-type nucleic acid (e.g., a nucleic acid encoding BE27) and a mutant form of this wild-type nucleic acid (e.g., a nucleic acid encoding a BE27 variant) may vary in sequence by the presence of one or more single base substitutions or by deletions and/or insertions of one or more nucleotides. These two forms of the nucleic acid are said to vary in sequence from one another. A second mutant form of the nucleic acid may exist. This second mutant form is said to vary in sequence from both the wild-type gene and the first mutant form of the nucleic acid.

As used herein, the terms “complementary”, “hybridizable”, or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., nucleotides that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine.

Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

It is understood in the art that the sequence of a polynucleotide need not be 100% complementary to that of its target nucleic acid to be hybridizable or specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, a nucleic acid in which 18 of 20 nucleotides of the nucleic acid are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular segments of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656, each of which is incorporated herein by reference) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489, incorporated herein by reference).

Thus, in some embodiments, “complementary” refers to a first nucleotide sequence that is at least 60%, 65%, 70%, 75%, 80%, 85%, 90%, 95%, 97%, 98%, or 99% identical to the complement of a second nucleotide sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleotides, or that the two sequences hybridize under stringent hybridization conditions. “Fully complementary” means each nucleotide of a first nucleic acid is capable of pairing with each nucleotide at a corresponding position in a second nucleic acid. For example, in certain embodiments, an oligonucleotide wherein each nucleotide has complementarity to a nucleic acid has a nucleotide sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleotides.

“Mismatch” means a nucleotide of a first nucleic acid that is not capable of pairing with a nucleotide at a corresponding position of a second nucleic acid.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960), each of which is incorporated herein by reference, have been followed by the refinement of this process into an essential tool of modern biology. For example, hybridization and washing conditions are now well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001), each of which is incorporated herein by reference. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) comprises a “double-stranded nucleic acid”. For example, triplex structures are considered to be “double-stranded”. In some embodiments, any base-paired nucleic acid is a “double-stranded nucleic acid”.

As used herein, the term “genomic locus” or “locus” (plural “loci”) is the specific location of a gene or DNA sequence on a chromosome.

As used herein, the term “gene” refers to a DNA nucleotide sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of technology described herein it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a nucleic acid (e.g., a gene or a gene product) that has the characteristics of that nucleic acid (e.g., a gene or a gene product) when isolated from a naturally occurring source. A wild-type nucleic acid (e.g., gene) is that which is most frequently observed in a population and is thus arbitrarily designated the “normal” or “wild-type” form of the nucleic acid (e.g., gene). In contrast, the term “modified,” “mutant,” or “polymorphic” refers to a nucleic acid (e.g., gene) or gene product that displays modifications in sequence and or functional properties (i.e., altered characteristics) when compared to the wild-type nucleic acid (e.g., gene) or gene product. It is noted that naturally-occurring mutants can be isolated; these are identified by the fact that they have altered characteristics when compared to the wild-type nucleic acid (e.g., gene) or gene product.

As used herein, the term “functional derivative” of a polypeptide is a compound having a qualitative biological property in common with said polypeptide. “Functional derivatives” include, but are not limited to, fragments of a polypeptide and derivatives of a polypeptide and its fragments, provided that they have a biological activity in common with a corresponding polypeptide. The term “derivative” encompasses both amino acid sequence variants of polypeptide, covalent modifications, and fusions thereof. A “fusion” polypeptide is a polypeptide comprising a polypeptide or portion (e.g., one or more domains) thereof fused or bonded to another heterologous polypeptide.

The terms “non-naturally occurring” or “engineered” are used interchangeably and indicate the involvement of the hand of man. The terms, when referring to nucleic acid molecules or polypeptides, mean that the nucleic acid molecule or the polypeptide is at least substantially free from at least one other component with which they are naturally associated in nature and as found in nature.

As used herein, the term “nuclease-deficient” refers to a protein comprising reduced nuclease activity, minimized nuclease activity (e.g., a nickase), undetectable nuclease activity, and/or having no nuclease activity, e.g., as a result of amino acid substitutions that reduce, minimize, and/or eliminate the nuclease activity of a protein. In some embodiments, a nuclease-deficient protein is described as a “dead” protein.

The term “oligonucleotide” as used herein is defined as a molecule comprising two or more deoxyribonucleotides or ribonucleotides, preferably at least 5 nucleotides, more preferably at least about 10 to 15 nucleotides and more preferably at least about 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The terms “peptide” and “polypeptide” and “protein” are used interchangeably herein and refer to a polymeric form of amino acids of any length, which can include coded and non-coded amino acids, chemically, or biochemically modified or derivatized amino acids, and polypeptides having modified peptide backbones. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

The term “binding”, as used herein (e.g., with reference to an RNA-binding domain of a polypeptide), refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, a DNA molecule (a DNA-binding protein), an RNA molecule (an RNA-binding protein), and/or a protein molecule (a proteinbinding protein). In the case of a protein domain-binding protein, it can bind to itself (to form homodimers, homotrimers, etc.) and/or it can bind to one or more molecules of a different protein or proteins.

As used herein, the term “ribonucleoprotein”, abbreviated “RNP” refers to a multimolecular complex comprising a polypeptide (e.g., a GEN-BE27 variant fusion (e.g., a Cas9 or Cas9-BE27 variant fusion protein, or a protein having an activity similar to a Cas9 or a Cas9-BE27 variant fusion protein (e.g., a Cpf1, Cpf1-BE27 variant fusion protein, or other Cas9-like protein, Cas9 homolog, and/or BE27 variant fusion thereof))) and a ribonucleic acid (e.g., a gRNA (e.g., sgRNA, a dgRNA)). In some embodiments, the polypeptide and ribonucleic acid are bound by a non-covalent interaction.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine/isoleucine, phenylalanine-tyrosine, lysine-arginine, alanine-valine, and asparagine-glutamine.

“Recombinant,” as used herein, means that a particular nucleic acid (DNA or RNA) is the product of various combinations of cloning, restriction, amplification (e.g., polymerase chain reaction (PCR)), and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may indeed act to modulate production of a desired product by various mechanisms). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid (e.g., to produce a BE27 variant). Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention but may be a naturally occurring amino acid sequence.

A “vector” or “expression vector” is a replicon, such as plasmid, phage, virus, or cosmid, to which another DNA segment, e.g., an “insert”, may be attached so as to bring about the replication of the attached segment in a cell.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam and Labhasetwar (2012), Advanced Drug Delivery Reviews, 64 (supplement): 61-71, incorporated herein by reference). The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995, incorporated herein by reference.

A “target nucleic acid” (e.g., a “target DNA”) as used herein is a polynucleotide (nucleic acid, gene, chromosome, genome, etc.) that comprises a “target site” or “target sequence.” The terms “target site” or “target sequence” are used interchangeably herein to refer to a nucleic acid sequence present in a target DNA to which a DNA-targeting segment of a DNA-targeting RNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand”.

The RNA molecule that binds to the polypeptide in the RNP and targets the polypeptide to a specific location within the target DNA is referred to herein as the “DNA targeting RNA” or “DNA-targeting RNA polynucleotide” (also referred to herein as a “guide RNA” or “gRNA”). A DNA-targeting RNA comprises two segments, a “DNA-targeting segment” and a “protein-binding segment.” In some embodiments, the gRNA comprises two RNAs (e.g., a dgRNA, e.g., a crRNA and a tracrRNA) and in some embodiments the gRNA comprises one RNA (e.g., a sgRNA).

By “segment” it is meant a segment or section or portion or region of a molecule, e.g., a contiguous segment of nucleotides in an RNA, DNA, or protein. A segment can also mean a segment or section or portion or region of a complex such that a segment may comprise regions of more than one molecule. For example, in some embodiments, the protein-binding segment (described below) of a DNA targeting RNA is one RNA molecule and the protein-binding segment therefore comprises a region of that RNA molecule. In other cases, the protein-binding segment (described below) of a DNA-targeting RNA comprises two separate molecules that are hybridized along a region of complementarity. As an illustrative, non-limiting example, a protein-binding segment of a DNA targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA (the complementary strand of the target DNA). The protein-binding segment (or “protein-binding sequence”) interacts with a polypeptide of the RNP. The protein-binding segment of a DNA-targeting RNA comprises two complementary segments of nucleotides that hybridize to one another to form a double stranded RNA duplex (dsRNA duplex).

A DNA-targeting RNA and a polypeptide form an RNP complex (e.g., bind via non-covalent interactions). The DNA-targeting RNA provides target specificity to the RNP complex by comprising a nucleotide sequence that is complementary to a sequence of a target DNA. The polypeptide of the RNP complex provides site-specific binding and, in some embodiments, a nuclease activity (e.g., for genome editing (e.g., by knockout, knockin, or other genomic and/or genetic modification)). In other words, the polypeptide of the RNP is guided to a target DNA sequence (e.g., a target sequence in a chromosomal nucleic acid; a target sequence in an extrachromosomal nucleic acid (e.g., an episomal nucleic acid, a minicircle, etc.); a target sequence in a mitochondrial nucleic acid; a target sequence in a chloroplast nucleic acid; a target sequence in a plasmid; etc.) by virtue of its association with the protein-binding segment of the DNA-targeting RNA.

In some embodiments, a DNA-targeting RNA comprises two separate RNA molecules (e.g., two RNA polynucleotides, e.g., an “activator-RNA” and a “targeter-RNA”) and is referred to herein as a “double-molecule DNA-targeting RNA” or a “two-molecule DNA-targeting RNA” or a “double guide RNA” or a “dgRNA”. In other embodiments, the DNA-targeting RNA is a single RNA molecule (e.g., a single RNA polynucleotide) and is referred to herein as a “single-molecule DNA-targeting RNA,” a “single guide RNA,” or an “sgRNA.” The term “DNA-targeting RNA” or “guide RNA” or “gRNA” is inclusive, referring both to double-molecule DNA-targeting RNAs (dgRNAs) and to single-molecule DNA-targeting RNAs (sgRNAs).

An exemplary two-molecule DNA-targeting RNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the DNA-targeting segment (single stranded) of the DNA-targeting RNA and a region (“duplex-forming segment”) that forms one half of the dsRNA duplex of the protein-binding segment of the DNA-targeting RNA. A corresponding tracrRNA-like molecule (activator-RNA) comprises a region (duplex-forming segment) that forms the other half of the dsRNA duplex of the protein-binding segment of the DNA-targeting RNA. In other words, a portion of the crRNA-like molecule is complementary to and hybridizes with a portion of a tracrRNA-like molecule to form the dsRNA duplex of the protein-binding domain of the DNA-targeting RNA. As such, each crRNA-like molecule can be said to have a corresponding tracrRNA-like molecule. The crRNA-like molecule additionally provides the single stranded DNA-targeting segment.

Thus, a crRNA-like molecule (e.g., a crRNA) and a tracrRNA-like molecule (e.g., a tracrRNA) hybridize (as a corresponding pair) to form a DNA-targeting RNA. The exact sequence of a given crRNA or tracrRNA molecule is characteristic of the species in which the RNA molecules are found. Various crRNAs and tracrRNAs are known in the art. A subject double molecule DNA-targeting RNA (dgRNA) can comprise any corresponding crRNA and tracrRNA pair. A subject double-molecule DNA-targeting RNA (sgRNA) can comprise any corresponding crRNA and tracrRNA pair.

The term “activator-RNA” is used herein to mean a tracrRNA-like molecule of a double molecule DNA-targeting RNA (e.g., a tracrRNA). The term “targeter-RNA” is used herein to mean a crRNA-like molecule of a double-molecule DNA-targeting RNA (e.g., a crRNA). The term “duplex-forming segment” is used herein to mean the segment of an activator-RNA or a targeter-RNA that contributes to the formation of the dsRNA duplex by hybridizing to a segment of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the DNA-targeting RNA. Therefore, a subject double-molecule DNA-targeting RNA can be comprised of any corresponding activator-RNA and targeter-RNA pair.

As used herein, “CRISPR system” refers collectively to transcripts and other elements involved in the expression of and/or directing the activity of CRISPR-associated (“Cas”) genes, including sequences encoding a Cas gene, dCas gene, Cas homolog, and/or Cpf1 gene; a tracr (trans-activating CRISPR) sequence (e.g., tracrRNA or an active partial tracrRNA); a cr (CRISPR) sequence (e.g., crRNA or an active partial crRNA); and/or other sequences and transcripts from a CRISPR locus. In some embodiments of the technology, the terms “guide sequence” and “guide RNA” (gRNA) are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR RNP complex (e.g., in vitro or in vivo) and direct it to the site of a target sequence in a cell (e.g., after introduction of the RNP).

As used herein, the terms “subject” and “patient” refer to any organisms including plants, microorganisms, and animals (e.g., mammals such as dogs, cats, livestock, and humans).

The terms “treatment”, “treating”, and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and In some embodiments after the symptomatic stage of the disease

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

The term “label” as used herein refers to any atom or molecule that can be used to provide a detectable (preferably quantifiable) effect, and that can be attached to a nucleic acid or protein. Labels include, but are not limited to, dyes (e.g., fluorescent dyes or moities); radiolabels such as ³²P; binding moieties such as biotin; haptens such as digoxigenin; luminogenic, phosphorescent, or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein, “moiety” refers to one of two or more parts into which something may be divided, such as, for example, the various parts of an oligonucleotide, a molecule, a chemical group, a domain, a probe, etc.

As used herein, a “stem-loop structure” refers to a nucleic acid having a secondary structure that includes a region of nucleotides that are known or predicted to form a double strand (stem portion) that is linked on one side to a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact basepairing. Thus, the stem may include one or more base mismatches. Alternatively, the basepairing may be exact, e.g., not include any mismatches.

As used herein, the term “homologous recombination” refers to a type of genetic recombination in which nucleotide sequences are exchanged between two similar or identical molecules of DNA known as homologous sequences or homology arms. Homologous recombination often involves the following basic steps: after a double-strand break (DSB) occurs on both strands of DNA, sections of DNA around the 5′ ends of the DSB are cut away in a process called resection. In the strand invasion step that follows, an overhanging 3′ end of the broken DNA molecule “invades” a similar or identical (or homologous) DNA molecule, e.g., a “homology arm”, that is not broken. After strand invasion, the further sequence of events may follow either of two main pathways—the DSBR (double-strand break repair) pathway or the SDSA (synthesis-dependent strand annealing) pathway.

As used herein, the term “endogenous genomic DNA” refers to a certain segment of genomic DNA, e.g., that is to be replaced by an insert by knockin. The endogenous genomic DNA, e.g., to be replaced or deleted, may or may not be homologous in sequence to the donor nucleic acid comprising the insert, so long as they are both flanked by the same or similar homology arms.

As used herein, the term “knockout” is a genetic modification resulting from the disruption of the genetic information encoded in a chromosomal locus.

As used herein, the term “knockin” is a genetic modification resulting from the replacement of the genetic information encoded in a chromosomal locus with a different nucleic acid sequence.

As used herein, the term “knockout organism” is an organism in which a significant proportion of the organism's cells harbor a knockout.

As used herein, the term “knockin organism” is an organism in which a significant proportion of the organism's cells harbor a knockin.

### DESCRIPTION

The use of gene editing tools (e.g., CRISPR/Cas9, TALEN, ZFN, etc., and related tools) has become a widely used gene editing technology. However, one aspect that remains to be improved is the low efficiency of knocking in (KI) large fragment nucleic acid inserts (e.g., a reporter gene) at a target site. For example, conventional CRISPR methods often have an efficiency of less than 1% for KI of large fragments. In addition, one important concern is off-target editing related to insufficient specificity of the CRISPR knock in.

### BE27 Variant Domains

In some embodiments, the present technology comprises use of a GEN-BE27 variant fusion in which a gene editing nuclease is fused to a modified exon 27 from the BR CA2 gene (e.g., a BE27 variant). See, e.g., the Examples herein. In some embodiments, a gene editing nuclease is fused to a plurality of BE27 variant domains (e.g., 1, 2, 3, 4, 5, 6, 7, 8, 9, 10, 11, 12, 13, 14, 15, 16, 17, 18, 19, 20, 21, 22, 23, 24, or 25 or more BE27 variant domains). In some embodiments, a polypeptide comprises a plurality of BE27 variant domains arranged serially, e.g., in a tandem array. In some embodiments, a GEN-BE27 variant fusion comprises a plurality of BE27 variant domains separated by one or more linker sequences (e.g., separating one or more of the plurality of the BE27 variant domains).

In some embodiments, the BE27 amino acid sequence is provided by human BRCA2 residues 3270 to 3305, e.g:

SEQ ID NO: 1
ALDFLSRLPLPPPVSPICTFVSPAAQKAFQPPRSCG

The BRCA2 gene sequence is provided by NCBI accession NG_012772.3, which is incorporated herein by reference. In some embodiments, the technology comprises a substituted variant of SEQ ID NO: 1 that provides the same or similar function of the BE27 polypeptide (e.g., SEQ ID NO: 1 comprising one or more conservative substitutions). In some embodiments, the technology comprises a substituted variant of SEQ ID NO: 1 that provides improved function of the BE27 polypeptide (e.g., SEQ ID NO: 1 comprising one or more substitutions), e.g., to provide a BE27 variant. In some embodiments, the BE27 variant is a substituted variant of the BE27 amino acid sequence comprising a substitution of the serine at position 15 (S15) and/or the serine at position 22 (S22) with another amino acid. In some embodiments, the BE27 variant is a substituted variant of the BE27 amino acid sequence comprising a substitution of alanine for the serine at position 15 (BE27-S15A), a substitution of tyrosine for the serine at position 22 (BE27-S22Y), or a substitution of aspartic acid for the serine at position 22 (BE27-S22D). The amino acid sequences of these particular exemplary BE27 variants are provided below:

BE27-S15A
(SEQ ID NO: 10)
A L D F L S R L P L P P P V A P I C T F V S P A A Q K A F Q P P R S C G
1 5 10 15 20 25 30 36
BE27-S22Y
(SEQ ID NO: 11)
A L D F L S R L P L P P P V S P I C T F V Y P A A Q K A F Q P P R S C G
1 5 10 15 20 25 30 36
BE27-S22D
(SEQ ID NO: 12)
A L D F L S R L P L P P P V S P I C T F V D P A A Q K A F Q P P R S C G
1 5 10 15 20 25 30 36

The technology includes any nucleic acid sequence encoding SEQ ID NOs: 1, 10, 11, or 12, e.g., a nucleic acid comprising SEQ ID NO: 7 and mutant forms thereof:

gcnytngayttyytnwsnmgnytnccnytnccnccnccngtnwsnccna
thtgyacnttygtnwsnccngcngcncaraargcnttycarccnccnmg
nwsntgyggn

In particular, the technology includes nucleic acids comprising a nucleotide sequence according to SEQ ID NO: 7 in which the codon for the serine at position 15 (S15) is mutated to produce a codon that codes for alanine (GCN) and/or in which the codon for the serine at position 22 (S22) is mutated to produce a codon that codes for tyrosine (TAY) or aspartic acid (GAY).

In some embodiments, the technology includes a nucleic acid having a nucleotide sequence that is at least 90% identical (e.g., at least 90, 91, 92, 93, 94, 95, 96, 97, 98, 99, 99.1, 99.2, 99.3, 99.4, 99.5, 99.6, 99.7, 99.8, or 99.9% identical) to the nucleotide sequence of SEQ ID NO: 7.

In some embodiments, a nucleic acid encoding a variant of the BE27 polypeptide (e.g., according to SEQ ID NO: 1) comprises a mutated form of SEQ ID NO: 8:

gccttggatttcttgagtagactgcctttacctccacctgttagtccca
tttgtacatttgtttctccggctgcacagaaggcatttcagccaccaag
gagttgtggc

In particular, the technology includes nucleic acids comprising a nucleotide sequence according to SEQ ID NO: 8 in which the codon for the serine at position 15 (S15) is mutated to produce a codon that codes for alanine (GCN) and/or in which the codon for the serine at position 22 (S22) is mutated to produce a codon that codes for tyrosine (TAY) or aspartic acid (GAY).

In some embodiments, the technology includes a nucleic acid having a nucleotide sequence that is at least 90% identical (e.g., at least 90, 91, 92, 93, 94, 95, 96, 97, 98, 99, 99.1, 99.2, 99.3, 99.4, 99.5, 99.6, 99.7, 99.8, or 99.9% identical) to the nucleotide sequence of SEQ ID NO: 8.

### RNP Complexes, Polypeptides, Ribonucleic Acids

In some embodiments, the technology comprises use of a ribonucleoprotein (RNP) comprising a GEN-BE27 variant fusion. In some embodiments, the technology comprises used of a RNP complex comprising a Cas9 or Cas9-like protein and an RNA (e.g., a gRNA (e.g., a subject DNA-targeting RNA, an activator-RNA and a targeter-RNA, a crRNA and a tracrRNA; a dgRNA; a sgRNA)). In some embodiments, the protein is a Cas9 or Cas9-like protein fused to a BE27 variant domain (“Cas9-BE27 variant” or “Cas9-BE27 variant fusion”) as described herein. Thus, in some embodiments, the technology comprises use of a ribonucleoprotein (RNP) complex comprising a Cas9 or Cas9-like protein fused to a BE27 variant domain (“Cas9-BE27 variant” or “Cas9-BE27 variant fusion”) as described herein and an RNA (e.g., e.g., a gRNA (e.g., a subject DNA-targeting RNA, an activator-RNA and a targeter-RNA, a crRNA and a tracrRNA; a dgRNA; a sgRNA)).

The RNA provides target specificity to the RNP complex by comprising a nucleotide sequence that is complementary to a sequence of a target DNA. The polypeptide of the complex provides binding and nuclease activity. In other words, the polypeptide is guided to a DNA sequence (e.g. a chromosomal sequence or an extrachromosomal sequence (e.g., an episomal sequence, a minicircle sequence, a mitochondrial sequence, a chloroplast sequence, etc.)) by virtue of its association with at least the protein-binding segment of the DNA-targeting RNA.

While various CRISPR/Cas systems have been used extensively for genome editing in cells of various types and species, recombinant and engineered nucleic acid-binding proteins such as Cas9 and Cas9-like proteins find use in the present technology to direct detectable labels to specific nucleic acids. Embodiments of the technology provide an RNP comprising a polypeptide, e.g., a Cas9, Cas9-BE27 variant fusion, or related or similar protein. The Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and destroys foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference).

Accordingly, some Cas9/RNA RNP complexes comprise two RNA molecules: (1) a CRISPR RNA (crRNA), possessing a nucleotide sequence complementary to the target nucleotide sequence; and (2) a trans-activating crRNA (tracrRNA). In this mode, Cas9 functions as an RNA-guided nuclease that uses both the crRNA and tracrRNA to recognize and cleave a target sequence. Recently, a single chimeric guide RNA (sgRNA) mimicking the structure of the annealed crRNA/tracrRNA has become more widely used than crRNA/tracrRNA because the gRNA approach provides a simplified system with only two components (e.g., the Cas9 or Cas9-BE27 variant fusion and the gRNA). Thus, sequence-specific binding of the RNP to a nucleic acid can be guided by a dual-RNA complex (e.g., a “dgRNA”), e.g., comprising a crRNA and a tracrRNA in two separate RNAs or by a chimeric single-guide RNA (e.g., a “sgRNA”) comprising a crRNA and a tracrRNA in a single RNA. (see, e.g., Jinek et al. (2012) “A Programmable Dual-RNA-Guided DNA Endonuclease in Adaptive Bacterial Immunity” Science 337:816-821, incorporated herein by reference).

As used herein, the targeting region of a crRNA (2-RNA dgRNA system) or a sgRNA (single guide system) is referred to as the “guide RNA” (gRNA). In some embodiments, the gRNA comprises, consists of, or essentially consists of 10 to 50 bases, e.g., 15 to 40 bases, e.g., 15 to 30 bases, e.g., 15 to 25 bases (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases). Methods are known in the art for determining the length of the gRNA that provides the most efficient target recognition for a Cas9. See, e.g., Lee et al. (2016) “The Neisseria meningitidis CRISPR-Cas9 System Enables Specific Genome Editing in Mammalian Cells” Molecular Therapy 24: 645 (2016), incorporated herein by reference.

Accordingly, in some embodiments, the gRNA is a short synthetic RNA comprising a “scaffold sequence” (protein-binding segment) for Cas9 or Cas9-BE27 binding and a user-defined “DNA-targeting sequence” (DNA-targeting segment) that is approximately 20-nucleotides long and is complementary to the target site of the target nucleic acid.

In some embodiments, DNA targeting specificity is determined by two factors: 1) a DNA sequence matching the gRNA targeting sequence and a protospacer adjacent motif (PAM) directly downstream of the target sequence. Some Cas9/gRNA complexes recognize a DNA sequence comprising a protospacer adjacent motif (PAM) sequence and an adjacent sequence comprising approximately 20 bases complementary to the gRNA. Canonical PAM sequences are NGG or NAG for Cas9 from Streptococcus pyogenes and NNNNGATT for the Cas9 from Neisseria meningitidis. In some embodiments, the technology comprises use of a Cas9 having an expanded PAM recognition (e.g., an xCas9 protein). Following DNA recognition by hybridization of the gRNA to the DNA target sequence, Cas9 cleaves the DNA sequence via an intrinsic nuclease activity. For genome editing and other purposes, the CRISPR/Cas system from S. pyogenes (“spCas9”) has been used most often. Using this system, one can target a given target nucleic acid (e.g., for editing or other manipulation) by designing a gRNA comprising a nucleotide sequence complementary to a DNA sequence (e.g., a DNA sequence comprising approximately 20 nucleotides) that is 5′-adjacent to the PAM. Methods are known in the art for determining a PAM sequence that provides efficient target recognition for a Cas9 (and thus for a Cas9-BE27). See, e.g., Zhang et al. (2013) “Processing-independent CRISPR RNAs limit natural transformation in Neisseria meningitidis” Molecular Cell 50: 488-503, incorporated herein by reference; Lee et al., supra, incorporated herein by reference.

In some exemplary embodiments, the crRNA comprises a sequence according to SEQ ID NO: 6

NNNNNNNNNNNNrGrUrUrUrArArGrArGrCrUrArUrGrCrUrGrUr
UrUrUrG

where the “NNNNNNNNNNNN” represents the DNA-targeting sequence that is complementary to the target sequence (e.g., of a nucleic acid to be subject to editing (e.g., knockin)). In some embodiments, the 5′ end of the crRNA comprises a detectable label, e.g., a dye, e.g., a fluorescent dye.

In some embodiments, the tracrRNA comprises a sequence of a naturally occurring tracrRNA, e.g., a provided by FIGS. 6, 35, and 37, and by SEQ ID NOs: 267-272 and 431-562 of U.S. pat. app. Pub. No. 20170051312, incorporated herein by reference.

In some embodiments, the crRNA comprises a sequence that hybridizes to a tracrRNA to form a duplex structure, e.g., a sequence provided by FIG. 7 and SEQ ID NOs: 563-679 of U.S. pat. app. Pub. No. 20170051312, incorporated herein by reference. In some embodiments, a crRNA comprises a sequence provided by FIG. 37 of U.S. pat. app. Pub. No. 20170051312, incorporated herein by reference. In some embodiments, the duplex-forming segment of the crRNA is at least about 60% identical to one of the tracrRNA molecules set forth in SEQ ID NOs: 431-679 of U.S. pat. app. Pub. No. 20170051312, incorporated herein by reference, or a complement thereof.

Thus, in some embodiments, exemplary (but not limiting) nucleotide sequences that are included in a dgRNA system include either of the sequences set forth in U.S. pat. app. Pub. No. 20170051312, incorporated herein by reference, as SEQ ID NOs: 431-562, or complements thereof pairing with any sequences set forth in U.S. pat. app. Pub. No. 20170051312, incorporated herein by reference, SEQ ID NOs: 563-679, or complements thereof that can hybridize to form a protein binding segment.

In some embodiments, a single-molecule gRNA (e.g., a sgRNA) comprises two complementary stretches of nucleotides that hybridize to form a dsRNA duplex. In some embodiments, the sgRNA (or a DNA encoding the sgRNA) is at least about 60% identical to one of the tracrRNA molecules set forth in U.S. pat. app. Pub. No. 20170051312, incorporated herein by reference, as SEQ ID NOs: 431-562, or a complement thereof, over at least 8 contiguous nucleotides. In some embodiments, the sgRNA (or a DNA encoding the sgRNA) is at least about 60% identical to one of the tracrRNA molecules set forth in U.S. pat. app. Pub. No. 20170051312, incorporated herein by reference, as SEQ ID NOs: 563-679, or a complement thereof, over at least 8 contiguous nucleotides. Appropriate naturally occurring pairs of crRNAs and tracrRNAs can be routinely determined by taking into account the species name and base-pairing (for the dsRNA duplex of the protein-binding domain) when determining appropriate cognate pairs.

In some embodiments, the technology provides a GEN-BE27 variant fusion that is a Cas9-BE27 variant fusion. In some embodiments, a Cas9-BE27 variant fusion/gRNA complex binds to a target nucleic acid with a sequence specificity provided by the gRNA to produce a double strand break in the nucleic acid. In some embodiments, the Cas9-BE27 variant fusion/gRNA RNP binds to the target nucleic acid with sequence specificity; in some embodiments, the RNP “melts” the target sequence to provide single-stranded regions of the target nucleic acid in a sequence-specific manner (see, e.g., Qi et al. (2013) “Repurposing CRISPR as an RNA-guided platform for sequence-specific control of gene expression” Cell 152(5): 1173-83, incorporated herein by reference).

Furthermore, while the Cas9/gRNA system initially targeted sequences adjacent to a PAM, in some embodiments the Cas9-BE27 variant fusion/gRNA system as used herein has been engineered to target any nucleotide sequence for binding (e.g., the technologies described herein are PAM-independent). Also, Cas9 orthologs encoded by compact genes (e.g., Cas9 from Staphylococcus aureus) are known (see, e.g., Ran et al. (2015) “In vivo genome editing using Staphylococcus aureus Cas9” Nature 520: 186-191, incorporated herein by reference), which improves the cloning and manipulation of the Cas9 components in vitro. The technology encompasses embodiments comprising use of these compact genes fused to BE27 variants described herein.

In some embodiments, different Cas9 proteins (e.g., Cas9 proteins from various species and modified versions (e.g., nuclease-deficient versions) thereof) may be advantageous to use in the various provided methods in order to capitalize on various characteristics of the different Cas9 proteins (e.g., for different PAM sequence preferences; for no PAM sequence requirement; for increased or decreased binding activity; for an increased or decreased level of cellular toxicity; for increase or decrease efficiency of in vitro RNP formation; for increase or decrease ability for introduction into cells (e.g., living cells, e.g., living primary cells), etc.). Cas9 proteins from various species may require different PAM sequences in the target DNA. Thus, for a particular Cas9 protein of choice, the PAM sequence requirement may be different than the 5′-XGG-3′ sequence described above. In some embodiments, the protein is an xCas protein having an expanded PAM compatibility (e.g., a Cas9 variant that recognizes a broad range of PAM sequences including NG, GAA and GAT), e.g., as described in Hu et al. (2018) “Evolved Cas9 variants with broad PAM compatibility and high DNA specificity” Nature 556: 57-63, incorporated herein by reference in its entirety.

In some embodiments, the technology comprises use of other RNA-guide nucleases (e.g., Cpf1 and modified versions thereof). For example, in some embodiments, use of other RNA-guide nucleases (e.g., Cpf1 and modified versions thereof) provides advantages—e.g., in some embodiments, the characteristics of the different nucleases are appropriate for methods as described herein (e.g., other RNA-guide nucleases have preferences for different PAM sequence preferences; other RNA-guide nucleases operate using single crRNAs other than cr/tracrRNA complexes; other RNA-guide nucleases operate with shorter guide RNAs, etc.) In some embodiments, the technology comprises use of a Cpf1 enzyme, e.g., as described in U.S. Pat. No. 9,790,490, which is incorporated herein by reference in its entirety.

Many Cas9 orthologs from a wide variety of species have been identified and the proteins share only a few identical amino acids. All identified Cas9 orthologs have the same domain architecture with a central HNH endonuclease domain and a split RuvC/RNaseH domain. Cas9 proteins share 4 key motifs with a conserved architecture. Motifs 1, 2, and 4 are RuvC like motifs and motif 3 is an HNH-motif. In some embodiments, a suitable polypeptide (e.g., a Cas9) comprises an amino acid sequence having 4 motifs, each of motifs 1-4 having at least approximately 75%, at least approximately 80%, at least approximately 85%, at least approximately 90%, at least approximately 95%, at least approximately 99%, or 100% amino acid sequence identity to the motifs 1-4 of a known Cas9 and/or Csn1 amino acid sequence.

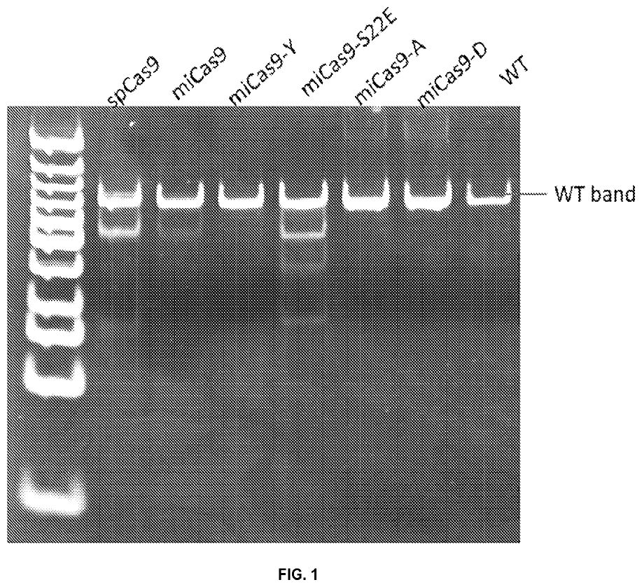

A number of bacteria express Cas9 protein variants. The Cas9 from Streptococcus pyogenes (spCas9) is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2- to 5-nucleotide sequence specified by the protein which is adjacent to the sequence specified by the RNA). Chylinski et al. classified Cas9 proteins from a large group of bacteria (RNA Biology 10: 5, 1-12; 2013, incorporated herein by reference), and a large number of Cas9 proteins are listed in supplementary FIG. 1 and supplementary table 1 thereof, which are incorporated by reference herein. Additional Cas9 proteins are described in Esvelt et al., Nat Methods. 2013 November; 10(11): 1116-21 and Fonfara et al., “Phylogeny of Cas9 determines functional exchangeability of dual-RNA and Cas9 among orthologous type II CRISPR-Cas systems.” Nucleic Acids Res. 42: 2577-90 (2014), each of which is incorporated herein by reference.

Cas9, and thus Cas9-BE27 variant fusions comprising Cas9, polypeptides of a variety of species find use in the technology described herein. While the S. pyogenes and S. thermophilus Cas9 molecules are widely used, Cas9 molecules of, derived from, or based on the Cas9 proteins of other species listed herein find use in embodiments of the technology. Accordingly, the technology provides for the replacement of S. pyogenes and S. thermophilus Cas9 and Cas9-BE27 variant fusions with Cas9 and Cas9-BE27 variant fusions from other species, e.g.:

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock 1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

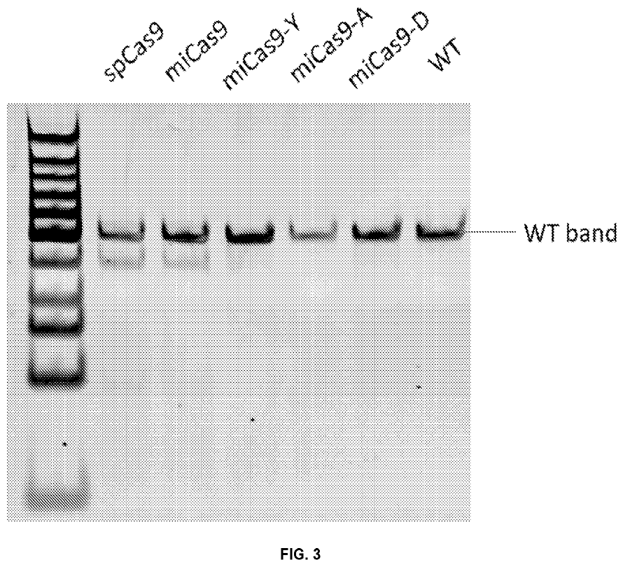

See also U.S. pat. app. Pub. No. 20170051312 at FIGS. 3, 4, 5, incorporated herein by reference.

In some embodiments, the technology described herein encompasses the use of a Cas9-BE27 variant fusion protein derived from any Cas9 protein (e.g., as listed above) and their corresponding guide RNAs or other guide RNAs that are compatible. The Cas9 from Streptococcus thermophilus LMD-9 CRISPR1 system has been shown to function in human cells (see, e.g., Cong et al. (2013) Science 339: 819, incorporated herein by reference). Additionally, Jinek showed in vitro that Cas9 orthologs from S. thermophilus and L. innocua, can be guided by a dual S. pyogenes gRNA to cleave target plasmid DNA.

In some embodiments, the present technology comprises the Cas9 protein from S. pyogenes (spCas9), either as encoded in bacteria or codon-optimized for expression in mammalian cells. For example, in some embodiments, the Cas9 used herein is at least approximately 50% identical to the sequence of S. pyogenes Cas9, e.g., at least 50% identical to the following sequence (SEQ ID NO: 9).

```
Met Asp Lys Lys Tyr Ser Ile Gly Leu Asp Ile Gly Thr Asn Ser Val
1 5 10 15
Gly Trp Ala Val Ile Thr Asp Glu Tyr Lys Val Pro Ser Lys Lys Phe
                        20 25 30
Lys Val Leu Gly Asn Thr Asp Arg His Ser Ile Lys Lys Asn Leu Ile
                35 40 45
Gly Ala Leu Leu Phe Asp Ser Gly Glu Thr Ala Glu Ala Thr Arg Leu
        50 55 60
Lys Arg Thr Ala Arg Arg Arg Tyr Thr Arg Arg Lys Asn Arg Ile Cys
65 70 75 80
Tyr Leu Gln Glu Ile Phe Ser Asn Glu Met Ala Lys Val Asp Asp Ser
                                85 90 95
Phe Phe His Arg Leu Glu Glu Ser Phe Leu Val Glu Glu Asp Lys Lys
                        100 105 110
His Glu Arg His Pro Ile Phe Gly Asn Ile Val Asp Glu Val Ala Tyr
                115 120 125
His Glu Lys Tyr Pro Thr Ile Tyr His Leu Arg Lys Lys Leu Val Asp
        130 135 140
Ser Thr Asp Lys Ala Asp Leu Arg Leu Ile Tyr Leu Ala Leu Ala His
145 150 155 160
Met Ile Lys Phe Arg Gly His Phe Leu Ile Glu Gly Asp Leu Asn Pro
                                165 170 175
Asp Asn Ser Asp Val Asp Lys Leu Phe Ile Gln Leu Val Gln Thr Tyr
                        180 185 190
Asn Gln Leu Phe Glu Glu Asn Pro Ile Asn Ala Ser Gly Val Asp Ala
                195 200 205
Lys Ala Ile Leu Ser Ala Arg Leu Ser Lys Ser Arg Arg Leu Glu Asn
        210 215 220
Leu Ile Ala Gln Leu Pro Gly Glu Lys Lys Asn Gly Leu Phe Gly Asn
225 230 235 240
Leu Ile Ala Leu Ser Leu Gly Leu Thr Pro Asn Phe Lys Ser Asn Phe
                                245 250 255
Asp Leu Ala Glu Asp Ala Lys Leu Gln Leu Ser Lys Asp Thr Tyr Asp
                        260 265 270
Asp Asp Leu Asp Asn Leu Leu Ala Gln Ile Gly Asp Gln Tyr Ala Asp
                275 280 285
Leu Phe Leu Ala Ala Lys Asn Leu Ser Asp Ala Ile Leu Leu Ser Asp
        290 295 300
Ile Leu Arg Val Asn Thr Glu Ile Thr Lys Ala Pro Leu Ser Ala Ser
305 310 315 320
Met Ile Lys Arg Tyr Asp Glu His His Gln Asp Leu Thr Leu Leu Lys
                                325 330 335
Ala Leu Val Arg Gln Gln Leu Pro Glu Lys Tyr Lys Glu Ile Phe Phe
                        340 345 350
Asp Gln Ser Lys Asn Gly Tyr Ala Gly Tyr Ile Asp Gly Gly Ala Ser
                355 360 365
Gln Glu Glu Phe Tyr Lys Phe Ile Lys Pro Ile Leu Glu Lys Met Asp
        370 375 380
Gly Thr Glu Glu Leu Leu Val Lys Leu Asn Arg Glu Asp Leu Leu Arg
385 390 395 400
Lys Gln Arg Thr Phe Asp Asn Gly Ser Ile Pro His Gln Ile His Leu
                                405 410 415
Gly Glu Leu His Ala Ile Leu Arg Arg Gln Glu Asp Phe Tyr Pro Phe
                        420 425 430
Leu Lys Asp Asn Arg Glu Lys Ile Glu Lys Ile Leu Thr Phe Arg Ile
                435 440 445
Pro Tyr Tyr Val Gly Pro Leu Ala Arg Gly Asn Ser Arg Phe Ala Trp
        450 455 460
Met Thr Arg Lys Ser Glu Glu Thr Ile Thr Pro Trp Asn Phe Glu Glu
465 470 475 480
Val Val Asp Lys Gly Ala Ser Ala Gln Ser Phe Ile Glu Arg Met Thr
                                485 490 495
Asn Phe Asp Lys Asn Leu Pro Asn Glu Lys Val Leu Pro Lys His Ser
                        500 505 510
Leu Leu Tyr Glu Tyr Phe Thr Val Tyr Asn Glu Leu Thr Lys Val Lys
                515 520 525
Tyr Val Thr Glu Gly Met Arg Lys Pro Ala Phe Leu Ser Gly Glu Gln
        530 535 540
Lys Lys Ala Ile Val Asp Leu Leu Phe Lys Thr Asn Arg Lys Val Thr
545 550 555 560
Val Lys Gln Leu Lys Glu Asp Tyr Phe Lys Lys Ile Glu Cys Phe Asp
                                565 570 575
Ser Val Glu Ile Ser Gly Val Glu Asp Arg Phe Asn Ala Ser Leu Gly
                        580 585 590
Thr Tyr His Asp Leu Leu Lys Ile Ile Lys Asp Lys Asp Phe Leu Asp
                595 600 605
Asn Glu Glu Asn Glu Asp Ile Leu Glu Asp Ile Val Leu Thr Leu Thr
        610 615 620
Leu Phe Glu Asp Arg Glu Met Ile Glu Glu Arg Leu Lys Thr Tyr Ala
625 630 635 640
His Leu Phe Asp Asp Lys Val Met Lys Gln Leu Lys Arg Arg Arg Tyr
                                645 650 655
Thr Gly Trp Gly Arg Leu Ser Arg Lys Leu Ile Asn Gly Ile Arg Asp
                        660 665 670
Lys Gln Ser Gly Lys Thr Ile Leu Asp Phe Leu Lys Ser Asp Gly Phe
                675 680 685
Ala Asn Arg Asn Phe Met Gln Leu Ile His Asp Asp Ser Leu Thr Phe
        690 695 700
Lys Glu Asp Ile Gln Lys Ala Gln Val Ser Gly Gln Gly Asp Ser Leu
705 710 715 720
His Glu His Ile Ala Asn Leu Ala Gly Ser Pro Ala Ile Lys Lys Gly
                                725 730 735
Ile Leu Gln Thr Val Lys Val Val Asp Glu Leu Val Lys Val Met Gly
                        740 745 750
Arg His Lys Pro Glu Asn Ile Val Ile Glu Met Ala Arg Glu Asn Gln
                755 760 765
Thr Thr Gln Lys Gly Gln Lys Asn Ser Arg Glu Arg Met Lys Arg Ile
        770 775 780
Glu Glu Gly Ile Lys Glu Leu Gly Ser Gln Ile Leu Lys Glu His Pro
785 790 795 800
Val Glu Asn Thr Gln Leu Gln Asn Glu Lys Leu Tyr Leu Tyr Tyr Leu
                                805 810 815
Gln Asn Gly Arg Asp Met Tyr Val Asp Gln Glu Leu Asp Ile Asn Arg
                        820 825 830
Leu Ser Asp Tyr Asp Val Asp His Ile Val Pro Gln Ser Phe Leu Lys
                835 840 845
Asp Asp Ser Ile Asp Asn Lys Val Leu Thr Arg Ser Asp Lys Asn Arg
        850 855 860
Gly Lys Ser Asp Asn Val Pro Ser Glu Glu Val Val Lys Lys Met Lys
865 870 875 880
Asn Tyr Trp Arg Gln Leu Leu Asn Ala Lys Leu Ile Thr Gln Arg Lys
                                885 890 895
Phe Asp Asn Leu Thr Lys Ala Glu Arg Gly Gly Leu Ser Glu Leu Asp
                        900 905 910
Lys Ala Gly Phe Ile Lys Arg Gln Leu Val Glu Thr Arg Gln Ile Thr
                915 920 925
Lys His Val Ala Gln Ile Leu Asp Ser Arg Met Asn Thr Lys Tyr Asp
        930 935 940
Glu Asn Asp Lys Leu Ile Arg Glu Val Lys Val Ile Thr Leu Lys Ser
945 950 955 960
Lys Leu Val Ser Asp Phe Arg Lys Asp Phe Gln Phe Tyr Lys Val Arg
                                965 970 975
Glu Ile Asn Asn Tyr His His Ala His Asp Ala Tyr Leu Asn Ala Val
                        980 985 990
Val Gly Thr Ala Leu Ile Lys Lys Tyr Pro Lys Leu Glu Ser Glu Phe
                995 1000 1005
Val Tyr Gly Asp Tyr Lys Val Tyr Asp Val Arg Lys Met Ile Ala
        1010 1015 1020
Lys Ser Glu Gln Glu Ile Gly Lys Ala Thr Ala Lys Tyr Phe Phe
        1025 1030 1035
Tyr Ser Asn Ile Met Asn Phe Phe Lys Thr Glu Ile Thr Leu Ala
        1040 1045 1050
Asn Gly Glu Ile Arg Lys Arg Pro Leu Ile Glu Thr Asn Gly Glu
        1055 1060 1065
Thr Gly Glu Ile Val Trp Asp Lys Gly Arg Asp Phe Ala Thr Val
        1070 1075 1080
Arg Lys Val Leu Ser Met Pro Gln Val Asn Ile Val Lys Lys Thr
        1085 1090 1095
Glu Val Gln Thr Gly Gly Phe Ser Lys Glu Ser Ile Leu Pro Lys
        1100 1105 1110
Arg Asn Ser Asp Lys Leu Ile Ala Arg Lys Lys Asp Trp Asp Pro
        1115 1120 1125
Lys Lys Tyr Gly Gly Phe Asp Ser Pro Thr Val Ala Tyr Ser Val
        1130 1135 1140
Leu Val Val Ala Lys Val Glu Lys Gly Lys Ser Lys Lys Leu Lys
        1145 1150 1155
Ser Val Lys Glu Leu Leu Gly Ile Thr Ile Met Glu Arg Ser Ser
        1160 1165 1170
Phe Glu Lys Asn Pro Ile Asp Phe Leu Glu Ala Lys Gly Tyr Lys
        1175 1180 1185
Glu Val Lys Lys Asp Leu Ile Ile Lys Leu Pro Lys Tyr Ser Leu
        1190 1195 1200
Phe Glu Leu Glu Asn Gly Arg Lys Arg Met Leu Ala Ser Ala Gly
        1205 1210 1215
Glu Leu Gln Lys Gly Asn Glu Leu Ala Leu Pro Ser Lys Tyr Val
        1220 1225 1230
Asn Phe Leu Tyr Leu Ala Ser His Tyr Glu Lys Leu Lys Gly Ser
        1235 1340 1245
Pro Glu Asp Asn Glu Gln Lys Gln Leu Phe Val Glu Gln His Lys
        1250 1255 1260
His Tyr Leu Asp Glu Ile Ile Glu Gln Ile Ser Glu Phe Ser Lys
        1265 1270 1275
Arg Val Ile Leu Ala Asp Ala Asn Leu Asp Lys Val Leu Ser Ala
        1280 1285 1290
Tyr Asn Lys His Arg Asp Lys Pro Ile Arg Glu Gln Ala Glu Asn
        1295 1300 1305
Ile Ile His Leu Phe Thr Leu Thr Asn Leu Gly Ala Pro Ala Ala
        1310 1315 1320
Phe Lys Tyr Phe Asp Thr Thr Ile Asp Arg Lys Arg Tyr Thr Ser
        1325 1330 1335
Thr Lys Glu Val Leu Asp Ala Thr Leu Ile His Gln Ser Ile Thr
        1340 1345 1350
Gly Leu Tyr Glu Thr Arg Ile Asp Leu Ser Gln Leu Gly Gly Asp
        1355 1360 1365
```

In some embodiments, the technology comprises use of a nucleotide sequence that is approximately 50%, 55%, 60%, 65%, 70%, 75%, 80%, 85%, 90%, 95%, 99%, or 100% identical to a nucleotide sequence that encodes a protein described by SEQ ID NO: 9.

In some embodiments, the Cas9 portion of the Cas9-BE27 variant fusion protein used herein is at least about 50% identical to the sequence of the S. pyogenes Cas9, e.g., at least 50%, 55%, 60%, 65%, 70%, 75%, 80%, 85%, 90%, 95%, 99% or 100% identical to SEQ ID NO: 9.

In some embodiments, the polypeptide (e.g., the RNA-guided nuclease) of the RNP is a Cas protein, CRISPR enzyme, or Cas-like protein. As used herein, the terms 50 “Cas protein”, “Cas9 protein”, “CRISPR enzyme”, and “Cas-like protein” include polypeptides, enzymatic activities, and polypeptides having activities similar to proteins known in the art as, or encoded by genes known in the art as, e.g., Cas1, Cas1B, Cas2, Cas3, Cas4, Cas5, Cas6, Cas7, Cas8, Cas9 (also known as Csn1 and Csx12), Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, homologs thereof, or modified versions thereof, e.g., including fusions of a BE27 variant with any of these Cas proteins, Cas9 proteins, CRISPR enzymes, and/or Cas-like proteins known in the art.

In embodiments, the technology comprises use of a polypeptide (e.g., a Type V/Type VI protein) such as Cpf1 or C2c1 or C2c2 and homologs and orthologs of a Type V/Type VI protein such as Cpf1 or C2c1 or C2c2 to provide a fusion with a BE27 variant. Embodiments encompass Cpf1, modified Cpf1 (e.g., Cpf1-BE27 variant fusion), and Cpf1, and CRISPR systems related to Cpf1, modified Cpf1 (Cpf1-BE27 variant fusion), and chimeric Cpf1. In some embodiments, the polypeptide (e.g., a Type V/Type VI protein) such as Cpf1 or C2c1 or C2c2 is from a genus that is, e.g., Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter; Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium, or Acidaminococcus. In some embodiments, a polypeptide (e.g., a Type V/Type VI protein) such as Cpf1 or C2c1 or C2c2 is from an organism that is, e.g., S. mutans, S. agalactiae, S. equisimilis, S. sanguinis, S. pneumonia; C. jejuni, C. coli; N. salsuginis, N. tergarcus; S. auricularis, S. carnosus; N meningitides, N gonorrhoeae; L. monocytogenes, L. ivanovii; C. botulinum, C. difficile, C. tetani, or C. sordellii. See, e.g., U.S. Pat. No. 9,790,490, incorporated herein by reference in its entirety. In some embodiments, a GEN-BE27 variant fusion comprises a Cpf1 protein and finds use as described in U.S. pat. app. Pub. No. 20180155716, which is incorporated herein by reference.

In some embodiments, differences from SEQ ID NO: 9 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11):1116-21 and Fonfara et al., Nucl. Acids Res. (2014) 42 (4): 2577-2590, each of which is incorporated herein by reference.

Thus, in some embodiments, the polypeptide of the Cas9 portion of the RNP is a naturally-occurring polypeptide. In some embodiments, the polypeptide of the Cas9 portion of the RNP is not a naturally-occurring polypeptide (e.g., a chimeric polypeptide, a naturally-occurring polypeptide that is modified, e.g., by one or more amino acid substitutions produced by an engineered nucleic acid comprising one or more nucleotide substitutions, deletions, insertions).

In some embodiments, choosing, designing, synthesizing, and analyzing nucleotide sequences and amino acid sequences (e.g., of the polypeptide and RNA components of an RNP complex as described herein) comprise use of sequence alignment methods to identify similarities and differences in two or more nucleotide sequences or amino acid sequences. To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence). The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch algorithm ((1970) J. Mol. Biol. 48:444-453, incorporated herein by reference), which has been incorporated into the GAP program in the GCG software package, e.g., using a Blosum 62 scoring matrix with a gap penalty of 12, a gap extend penalty of 4, and a frameshift gap penalty of 5. Other methods are known in the art, e.g., as discussed elsewhere herein.

In some embodiments, the RNP comprises a protein that is a Cas9 or Cas9 derivative, e.g., a Cas9-BE27 variant fusion. Thus, in some embodiments, the protein is a Type II Cas9 protein. In some embodiments, the Cas9 has been engineered to partially remove the nuclease domain (e.g., a “dead Cas9” or a “Cas9 nickase”; see, e.g., Nature Methods 11: 399-402 (2014), incorporated herein by reference). In some embodiments, the RNP protein is a protein from a CRISPR system other than the S. pyogenes system, e.g., a Type V Cpf1, C2c1, C2c2, or C2c3 protein, or a derivative of one of the foregoing.

In some embodiments, the polypeptide of the RNP is a chimeric or fusion polypeptide, e.g., a polypeptide that comprises two or more functional domains (e.g., a Cas9 and a BE27 variant domain). For example, in some embodiments a chimeric polypeptide interacts with (e.g., binds to) an RNA to form an RNP (described above). The RNA guides the polypeptide to a target sequence within target DNA (e.g. a chromosomal sequence or an extrachromosomal sequence, e.g. an episomal sequence, a minicircle sequence, a mitochondrial sequence, a chloroplast sequence, etc.). Thus, in some embodiments a chimeric polypeptide binds target DNA.

A chimeric or fusion polypeptide comprises at least two portions, e.g., an RNA binding portion and an “activity” portion (e.g., a label). A chimeric or fusion polypeptide comprises amino acid sequences that are derived from at least two different polypeptides. A chimeric or fusion polypeptide can comprise modified and/or naturally occurring polypeptide sequences (e.g., a first amino acid sequence from a modified or unmodified Cas9 protein; and a second amino acid sequence other than the Cas9 protein, e.g., a BE27 variant domain).

In some embodiments, the RNA-binding portion of a chimeric polypeptide is a naturally-occurring polypeptide. In some embodiments, the RNA-binding portion of a chimeric polypeptide is not a naturally-occurring molecule (e.g., modified with respect to a naturally-occurring polypeptide by, e.g., substitution, deletion, and/or insertion). In some embodiments, naturally-occurring RNA-binding portions of interest are derived from polypeptides known in the art, e.g., discussed herein (e.g., Cas9 and similar polypeptides).

In some embodiments, the RNA-binding portion of a chimeric polypeptide comprises an amino acid sequence having at least approximately 75%, at least approximately 80%, at least approximately 85%, at least approximately 90%, at least approximately 95%, at least approximately 98%, at least approximately 99%, or 100% amino acid sequence identity to the RNA-binding portion of a polypeptide described herein.

In some embodiments, the chimeric polypeptide comprises an amino acid sequence having at least approximately 75%, at least approximately 80%, at least approximately 85%, at least approximately 90%, at least approximately 95%, at least approximately 99%, or 100% amino acid sequence identity to a portion of a Cas9 amino acid sequence provided herein.

In addition to the RNA-binding portion, the chimeric polypeptide comprises an “activity portion”, e.g., a BE27 variant domain.

A gRNA comprises a first segment (also referred to herein as a “DNA-targeting segment” or a “DNA-targeting sequence”) and a second segment (also referred to herein as a “protein-binding segment” or a “protein-binding sequence”).

The DNA-targeting segment of a gRNA comprises a nucleotide sequence that is complementary to a sequence in a target DNA. In other words, the DNA-targeting segment of a gRNA interacts with a target DNA in a sequence-specific manner via hybridization (e.g., complementary base pairing). As such, the nucleotide sequence of the DNA targeting segment may vary and determines the location within the target DNA that the DNA targeting RNA and the target DNA will interact. The DNA-targeting segment of a gRNA can be modified (e.g., by genetic engineering) to hybridize to any desired sequence within a target DNA.

The DNA-targeting segment (e.g., comprising the DNA-targeting sequence and, in some embodiments, additional nucleic acid) can have a length of from approximately 8 nucleotides to approximately 100 nucleotides. For example, the DNA-targeting segment can have a length of from approximately 12 nucleotides (nt) to approximately 80 nt, from approximately 12 nt to approximately 50 nt, from approximately 12 nt to approximately 40 nt, from approximately 12 nt to approximately 30 nt, from approximately 12 nt to approximately 25 nt, from approximately 12 nt to approximately 20 nt, or from approximately 12 nt to approximately 19 nt. For example, the DNA-targeting segment can have a length of from approximately 19 nt to approximately 20 nt, from approximately 19 nt to approximately 25 nt, from approximately 19 nt to approximately 30 nt, from approximately 19 nt to approximately 35 nt, from approximately 19 nt to approximately 40 nt, from approximately 19 nt to approximately 45 nt, from approximately 19 nt to approximately 50 nt, from approximately 19 nt to approximately 60 nt, from approximately 19 nt to approximately 70 nt, from approximately 19 nt to approximately 80 nt, from approximately 19 nt to approximately 90 nt, from approximately 19 nt to approximately 100 nt, from approximately 20 nt to approximately 25 nt, from approximately 20 nt to approximately 30 nt, from approximately 20 nt to approximately 35 nt, from approximately 20 nt to approximately 40 nt, from approximately 20 nt to approximately 45 nt, from approximately 20 nt to approximately 50 nt, from approximately 20 nt to approximately 60 nt, from approximately 20 nt to approximately 70 nt, from approximately 20 nt to approximately 80 nt, from approximately 20 nt to approximately 90 nt, or from approximately 20 nt to approximately 100 nt.

In some embodiments, the nucleotide sequence (the DNA-targeting sequence) of the DNA-targeting segment that is complementary to a nucleotide sequence (target sequence) of the target DNA can have a length at least approximately 12 nt. For example, the DNA-targeting sequence of the DNA-targeting segment that is complementary to a target sequence of the target DNA can have a length at least approximately 12 nt, at least approximately 15 nt, at least approximately 18 nt, at least approximately 19 nt, at least approximately 20 nt, at least approximately 25 nt, at least approximately 30 nt, at least approximately 35 nt or at least approximately 40 nt. For example, the DNA-targeting sequence of the DNA-targeting segment that is complementary to a target sequence of the target DNA can have a length of from approximately 12 nucleotides (nt) to approximately 80 nt, from approximately 12 nt to approximately 50 nt, from approximately 12 nt to approximately 45 nt, from approximately 12 nt to approximately 40 nt, from approximately 12 nt to approximately 35 nt, from approximately 12 nt to approximately 30 nt, from approximately 12 nt to approximately 25 nt, from approximately 12 nt to approximately 20 nt, from approximately 12 nt to approximately 19 nt, from approximately 19 nt to approximately 20 nt, from approximately 19 nt to approximately 25 nt, from approximately 19 nt to approximately 30 nt, from approximately 19 nt to approximately 35 nt, from approximately 19 nt to approximately 40 nt, from approximately 19 nt to approximately 45 nt, from approximately 19 nt to approximately 50 nt, from approximately 19 nt to approximately 60 nt, from approximately 20 nt to approximately 25 nt, from approximately 20 nt to approximately 30 nt, from approximately 20 nt to approximately 35 nt, from approximately 20 nt to approximately 40 nt, from approximately 20 nt to approximately 45 nt, from approximately 20 nt to approximately 50 nt, or from approximately 20 nt to approximately 60 nt. The nucleotide sequence (the DNA-targeting sequence) of the DNA-targeting segment that is complementary to a nucleotide sequence (target sequence) of the target DNA can have a length at least approximately 12 nt.

In additional embodiments, the nucleotide sequence (the DNA-targeting sequence) of the DNA-targeting segment that is complementary to a nucleotide sequence (target sequence) of the target DNA can have a length of from approximately 8 nucleotides to approximately 30 nucleotides. For example, the DNA-targeting segment can have a length of from approximately 8 nucleotides (nt) to approximately 30 nt, from approximately 8 nt to approximately 30 nt, from approximately 8 nt to approximately 25 nt, from approximately 8 nt to approximately 20 nt, from approximately 8 nt to approximately 18 nt, from approximately 8 nt to approximately 15 nt, or from approximately 8 nt to approximately 12 nt, e.g., 8 nt, 9 nt, 10 nt, 11 nt, or 12 nt.

In some embodiments, the DNA-targeting sequence of the DNA-targeting segment that is complementary to a target sequence of the target DNA is 8-20 nucleotides in length. In some embodiments, the DNA-targeting sequence of the DNA-targeting segment that is complementary to a target sequence of the target DNA is 9-12 nucleotides in length.

The percent complementarity between the DNA-targeting sequence of the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the DNA-targeting segment and the target sequence of the target DNA is 100% over the seven contiguous 5′-most nucleotides of the target sequence of the complementary strand of the target DNA. In some embodiments, the percent complementarity between the DNA-targeting sequence of the DNA-targeting segment and the target sequence of the target DNA is at least 60% over approximately 20 contiguous nucleotides. In some embodiments, the percent complementarity between the DNA-targeting sequence of the DNA-targeting segment and the target sequence of the target DNA is 100% over the fourteen contiguous 5′-most nucleotides of the target sequence of the complementary strand of the target DNA and as low as 0% over the remainder. In such a case, the DNA-targeting sequence can be considered to be 14 nucleotides in length. In some embodiments, the percent complementarity between the DNA targeting sequence of the DNA-targeting segment and the target sequence of the target DNA is 100% over the seven contiguous 5′-most nucleotides of the target sequence of the complementary strand of the target DNA and as low as 0% over the remainder. In such a case, the DNA-targeting sequence can be considered to be 7 nucleotides in length.

The protein-binding segment of a gRNA interacts with a polypeptide, e.g., a Cas9, Cas9-BE27 variant fusion, or a Cas9-like protein-BE27 variant fusion. The gRNA guides the bound polypeptide to a specific nucleotide sequence within target DNA via the above mentioned DNA-targeting segment. The protein-binding segment of a gRNA comprises two segments comprising nucleotide sequences that are complementary to one another. The complementary nucleotides of the protein-binding segment hybridize to form a double stranded RNA duplex.

A dgRNA comprises two separate RNA molecules. Each of the two RNA molecules of a dgRNA comprises a segment is complementary to one another such that the complementary nucleotides of the two RNA molecules hybridize to form the double stranded RNA duplex of the protein-binding segment.

In some embodiments, the duplex-forming segment of the activator-RNA is at least approximately 60% identical to one of the activator-RNA (tracrRNA) molecules set forth in U.S. pat. app. Pub. No. 20170051312, incorporated herein by reference, as SEQ ID NOs: 431-562, or a complement thereof, over a segment of at least 8 contiguous nucleotides. For example, the duplex-forming segment of the activator-RNA (or the DNA encoding the duplex-forming segment of the activator-RNA) is at least approximately 60% identical, at least approximately 65% identical, at least approximately 70% identical, at least approximately 75% identical, at least approximately 80% identical, at least approximately 85% identical, at least approximately 90% identical, at least approximately 95% identical, at least approximately 98% identical, at least approximately 99% identical, or 100% identical, to one of the tracrRNA sequences set forth in U.S. pat. app. Pub. No. 20170051312, incorporated herein by reference, as SEQ ID NOs: 431-562, or a complement thereof, over a segment of at least 8 contiguous nucleotides.

In some embodiments, the duplex-forming segment of the targeter-RNA is at least approximately 60% identical to one of the targeter-RNA (crRNA) sequences set forth in U.S. pat. app. Pub. No. 20170051312, incorporated herein by reference, as SEQ ID NOs: 563-679, or a complement thereof, over a segment of at least 8 contiguous nucleotides. For example, the duplex-forming segment of the targeter-RNA (or the DNA encoding the duplex-forming segment of the targeter-RNA) is at least approximately 65% identical, at least approximately 70% identical, at least approximately 75% identical, at least approximately 80% identical, at least approximately 85% identical, at least approximately 90% identical, at least approximately 95% identical, at least approximately 98% identical, at least approximately 99% identical, or 100% identical to one of the crRNA sequences set forth in U.S. pat. app. Pub. No. 20170051312, incorporated herein by reference, as SEQ ID NOs: 563-679, or a complement thereof, over a segment of at least 8 contiguous nucleotides.

Non-limiting examples of nucleotide sequences that can be included in a two-molecule DNA targeting RNA (dgRNA) include either of the sequences set forth in U.S. pat. app. Pub. No. 20170051312, incorporated herein by reference, as SEQ ID NOs: 431-562, or complements thereof pairing with any sequences set forth in U.S. pat. app. Pub. No. 20170051312, incorporated herein by reference, as SEQ ID NOs: 563-679, or complements thereof that can hybridize to form a protein binding segment.

A single-molecule DNA-targeting RNA (sgRNA) comprises two segments of nucleotides (a targeter-RNA and an activator-RNA) that are complementary to one another, are covalently linked by intervening nucleotides (“linkers” or “linker nucleotides”), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The linker of a single-molecule DNA-targeting RNA can have a length of from approximately 3 nucleotides to approximately 100 nucleotides. For example, the linker can have a length of from approximately 3 nucleotides (nt) to approximately 90 nt, from approximately 3 nucleotides (nt) to approximately 80 nt, from approximately 3 nucleotides (nt) to approximately 70 nt, from approximately 3 nucleotides (nt) to approximately 60 nt, from approximately 3 nucleotides (nt) to approximately 50 nt, from approximately 3 nucleotides (nt) to approximately 40 nt, from approximately 3 nucleotides (nt) to approximately 30 nt, from approximately 3 nucleotides (nt) to approximately 20 nt or from approximately 3 nucleotides (nt) to approximately 10 nt. For example, the linker can have a length of from approximately 3 nt to approximately 5 nt, from approximately 5 nt to approximately 10 nt, from approximately 10 nt to approximately 15 nt, from approximately 15 nt to approximately 20 nt, from approximately 20 nt to approximately 25 nt, from approximately 25 nt to approximately 30 nt, from approximately 30 nt to approximately 35 nt, from approximately 35 nt to approximately 40 nt, from approximately 40 nt to approximately 50 nt, from approximately 50 nt to approximately 60 nt, from approximately 60 nt to approximately 70 nt, from approximately 70 nt to approximately 80 nt, from approximately 80 nt to approximately 90 nt, or from approximately 90 nt to approximately 100 nt. In some embodiments, the linker of a single molecule DNA-targeting RNA is 4 nt.

An exemplary single-molecule DNA-targeting RNA comprises two complementary segments of nucleotides that hybridize to form a dsRNA duplex. In some embodiments, one of the two complementary segments of nucleotides of the single-molecule DNA-targeting RNA (or the DNA encoding the segment) is at least approximately 60% identical to one of the activator-RNA (tracrRNA) molecules set forth in U.S. pat. app. Pub. No. 20170051312, incorporated herein by reference, as SEQ ID NOs: 431-562, or a complement thereof, over a segment of at least 8 contiguous nucleotides. For example, one of the two complementary segments of nucleotides of the single-molecule DNA-targeting RNA (or the DNA encoding the segment) is at least approximately 65% identical, at least approximately 70% identical, at least approximately 75% identical, at least approximately 80% identical, at least approximately 85% identical, at least approximately 90% identical, at least approximately 95% identical, at least approximately 98% identical, at least approximately 99% identical, or 100% identical to one of the tracrRNA sequences set forth in U.S. pat. app. Pub. No. 20170051312, incorporated herein by reference, as SEQ ID NOs: 431-562, or a complement thereof, over a segment of at least 8 contiguous nucleotides.

In some embodiments, one of the two complementary segments of nucleotides of the single molecule DNA-targeting RNA (or the DNA encoding the segment) is at least approximately 60% identical to one of the targeter-RNA (crRNA) sequences set forth in U.S. pat. app. Pub. No. 20170051312, incorporated herein by reference, as SEQ ID NOs: 563-679, or a complement thereof, over a segment of at least 8 contiguous nucleotides. For example, one of the two complementary segments of nucleotides of the single-molecule DNA-targeting RNA (or the DNA encoding the segment) is at least approximately 65% identical, at least approximately 70% identical, at least approximately 75% identical, at least approximately 80% identical, at least approximately 85% identical, at least approximately 90% identical, at least approximately 95% identical, at least approximately 98% identical, at least approximately 99% identical, or 100% identical to one of the crRNA sequences set forth in U.S. pat. app. Pub. No. 20170051312, incorporated herein by reference, as SEQ ID NOs: 563-679, or a complement thereof, over a stretch of at least 8 contiguous nucleotides.

With regard to both sgRNA and dgRNA, the technology comprises artificial nucleotide sequences that share a wide range of identity (approximately at least 50% identity) with naturally occurring tracrRNAs and crRNAs that function with Cas9 and Cas9-BE27 variant fusions to deliver RNP to target nucleic acids with sequence specificity, particularly because the structure of the protein-binding domain of the DNA-targeting RNA is conserved. Thus, information and modeling relating to RNA folding and RNA secondary structure of a naturally occurring protein-binding domain of a DNA-targeting RNA provides guidance to design artificial protein-binding domains (either in dgRNA or sgRNA). As a non-limiting example, a functional artificial DNA-targeting RNA may be designed based on the structure of the protein-binding segment of a naturally occurring DNA-targeting segment of an RNA (e.g., including the same or similar number of base pairs along the RNA duplex and including the same or similar “bulge” region as present in the naturally occurring RNA). Structures can readily be produced by one of ordinary skill in the art for any naturally occurring crRNA:tracrRNA pair from any species; thus, in some embodiments an artificial DNA-targeting-RNA is designed to mimic the natural structure for a given species when using a Cas9 (or a related Cas9) from that species. Thus, in some embodiments a suitable DNA-targeting RNA is an artificially designed RNA (non-naturally occurring RNA) comprising a protein-binding domain that was designed to mimic the structure of a protein-binding domain of a naturally occurring DNA-targeting RNA. In exemplary embodiments, the protein-binding segment has a length of from approximately 10 nucleotides to approximately 100 nucleotides; e.g., the protein-binding segment has a length of from approximately 15 nucleotides (nt) to approximately 80 nt, from approximately 15 nt to approximately 50 nt, from approximately 15 nt to approximately 40 nt, from approximately 15 nt to approximately 30 nt or from approximately 15 nt to approximately 25 nt.

Nucleic acids can be analyzed and designed using a variety of computer tools, e.g., Vector NTI (Invitrogen) for nucleic acids and AlignX for comparative sequence analysis of proteins. Further, in silico modeling of RNA structure and folding can be performed using the Vienna RNA package algorithms and RNA secondary structures and folding models can be predicted with RNAfold and RNAcofold, respectively, and visualized with VARNA. See, e.g., Denman (1993), Biotechniques 15, 1090; Hofacker and Stadler (2006), Bioinformatics 22, 1172; and Darty and Ponty (2009), Bioinformatics 25, 1974, each of which is incorporated herein by reference.

Thus, as described herein, in some embodiments, the technology provides methods, systems, kits, compositions, reaction mixtures, uses, etc. comprising and/or comprising use of an RNP comprising a polypeptide and one or more RNAs. In some embodiments, the RNA comprises a segment (e.g., comprising 6-10 nucleotides, e.g., comprising 6, 7, 8, 9, or 10 nucleotides) that is complementary (e.g., at least 80, 81, 82, 83, 84, 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, 99.9, or 100% complementary) to a nucleotide sequence in the target DNA.

In some embodiments, the RNA comprises a segment comprising a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 431-682 (e.g., SEQ ID NOs: 431-562) of U.S. pat. app. Pub. No. 20170051312, incorporated herein by reference. In some embodiments, the RNA comprises a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 563-682 of U.S. pat. app. Pub. No. 20170051312, incorporated herein by reference.

In some embodiments, the polypeptide comprises a segment comprising an amino acid sequence that is at least approximately 75% amino acid identical to amino acids 7-166 or 731-1003 of any of the amino acid sequences set forth as SEQ ID NOs: 1-256 and 795-1346 of U.S. pat. app. Pub. No. 20170051312, incorporated herein by reference.

In some embodiments, the technology comprises use of an RNA-targeting protein (e.g., Cas13 and Cas13-BE27 variant fusions), which works according to a similar mechanism as Cas9 and Cas9-BE27 variant fusions. In addition to targeting genomic DNA, Cas9 and other CRISPR related proteins (e.g., Cas13) also target RNAs directed by gRNAs (see, e.g., Abudayyeh et al. (2017) “RNA targeting with CRISPR-Cas13” Nature 550: 280, incorporated herein by reference). Thus, in some embodiments, gRNAs complex with Cas9 or other RNA-guided nucleases (e.g., a class 2 type VI RNA-guided RNA-targeting CRISPR-Cas effector (e.g., Cas13), a Cpf1, etc.) fused to a BE27 variant domain to edit RNA transcripts and non-coding RNAs in cells. Accordingly, in some embodiments, the technology relates to targeting RNAs using guide RNAs in complex with a Cas9-BE27 variant fusion or an RNA-targeting Cas13-BE27 variant fusion.

### Donor Nucleic Acid

In some embodiments, the technology comprises use of a donor nucleic acid, e.g., a DNA molecule. In some embodiments, the donor molecule participates in HDR to “repair” the DSB with a sequence from the donor. In this way, CRISPR finds use to make targeted insertions of a particular nucleic acid sequence at a target site, e.g., to produce knock-ins (KI).

In some embodiments, the donor nucleic acid is double-stranded. In some embodiments, the donor nucleic acid is single-stranded. In some embodiments, a donor DNA molecule is a linear molecule (e.g., not a circular molecule such as a plasmid DNA).

A donor DNA molecule can have any desired sequence. In some embodiments, the donor nucleic acid comprises a portion comprising a nucleic acid to be knocked-in at a target locus (e.g., in some embodiments, the donor nucleic acid comprises a portion comprising an insertion sequence). In some embodiments, the 3′ most nucleotide on at least one end of the donor DNA molecule is a C. In some embodiments, the 3′ most nucleotide on one and only one end of the donor DNA molecule is a C. In some embodiments, the 3′ most nucleotide on at least one end of the donor DNA molecule is a G. In some embodiments, the 3′ most nucleotide on one and only one end of the donor DNA molecule is a G. In some embodiments, the 3′ most nucleotide on at least one end of the donor DNA molecule is an A. In some embodiments, the 3′ most nucleotide on one and only one end of the donor DNA molecule is an A. In some embodiments, the 3′ most nucleotide on at least one end of the donor DNA molecule is a T. In some embodiments, the 3′ most nucleotide on one and only one end of the donor DNA molecule is a T.

In some embodiments, the linear donor (e.g., DNA) molecule has a length in a range of from 10 to 1000 nucleotides (nt) (e.g., 15 to 500, 20 to 500, 30 to 500, 33 to 500, 35 to 500, 40 to 500, 45 to 500, 50 to 500, 15 to 250, 20 to 250, 30 to 250, 33 to 250, 35 to 250, 40 to 250, 45 to 250, 50 to 250, 15 to 150, 20 to 150, 30 to 150, 33 to 150, 35 to 150, 40 to 150, 45 to 150, 50 to 150, 15 to 100, 20 to 100, 30 to 100, 33 to 100, 35 to 100, 40 to 100, 45 to 100, 50 to 100, 15 to 50, 20 to 50, 30 to 50, 33 to 50, 35 to 50, 40 to 50, or 45 to 50 nt). In some embodiments, the linear donor nucleic acid has a length of 1 Kbp or more (e.g., 1 to 10 Kbp (e.g., 1, 1.5, 2, 2.5, 3, 3.5, 4, 4.5, 5, 5.5, 6, 6.5, 7, 7.5, 8, 8.5, 9, 9.5, or 10 Kbp).

In some embodiments, a method provided herein includes introducing into a cell a subject linear donor DNA molecule. In some embodiments, a donor DNA molecule includes a label (e.g., as defined above, e.g., a biotin label, a fluorescent dye, etc.)

In some embodiments, the linear donor DNA molecule includes a 3′-overhang. For example, In some embodiments, the linear donor DNA molecule includes a 3′-overhang having a length in a range of from 1 to 6 nucleotides (nt) (e.g., 1 to 5 nt, 1 to 4 nt, 1 to 3 nt, 1 to 2 nt, 2 to 6 nt, 2 to 5 nt, 2 to 4 nt, 2 to 3 nt, 3 to 6 nt, 3 to 5 nt, 3 to 4 nt, 4 to 6 nt, 4 to 5 nt, 5 to 6 nt, 1 nt, 2 nt, 3 nt, 4 nt, 5 nt, or 6 nt). In some embodiments, the linear donor DNA molecule does not have a 3-overhang. Thus, In some embodiments, the linear donor DNA molecule includes a 3-overhang having a length in a range of from 0 to 6 nucleotides (nt) (e.g., 0 to 5 nt, 0 to 4 nt, 0 to 3 nt, 0 to 2 nt, 0 to 1 nt, 1 to 6 nt, 1 to 5 nt, 1 to 4 nt, 1 to 3 nt, 1 to 2 nt, 2 to 6 nt, 2 to 5 nt, 2 to 4 nt, 2 to 3 nt, 3 to 6 nt, 3 to 5 nt, 3 to 4 nt, 4 to 6 nt, 4 to 5 nt, 5 to 6 nt, 1 nt, 2 nt, 3 nt, 4 nt, 5 nt, or 6 nt).

### Synthesis and Assembly of RNP and Delivery of RNP

In some embodiments, the Cas9-BE27 variant fusion protein is synthesized, purified, and assembled in vitro. In some embodiments, the gRNA is transcribed in vitro. In some embodiments, the gRNA is chemically synthesized de novo. In some embodiments, the RNP complex is assembled in vitro using in vitro-transcribed, or de novo-synthesized single guide RNA (sgRNA) and a protein that is synthesized, purified, and folded in vitro.

In some embodiments, an expression system (e.g., comprising an expression vector and a suitable expression host) finds use in producing a polypeptide and/or an RNA of the RNP. Numerous suitable expression vectors are known to those of skill in the art, and many are commercially available. The following vectors are provided by way of example for eukaryotic host cells, e.g., pXT1, pSG5 (Stratagene), pSVK3, pBPV, pMSG, and pSVLSV40 (Pharmacia). However, any other vector may be used if it is compatible with the host cell. Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544, incorporated herein by reference).

In some embodiments, the protein is provided as a single polypeptide (e.g., a full GEN-BE27 variant fusion). In some embodiments, the protein is provided in multiple polypeptides, e.g., a split GEN-BE27 variant fusion protein provided in two parts, three parts, etc.

In some embodiments, the RNP is provided as a nanoparticle for administration to a live organism.

In some embodiments, the RNP is delivered into cells using a technique or composition related to nucleofection, cell penetrating peptide, viral vesicles, cell surface tunneling protein, ultrasound, electroporation, cell squeezing, nanoparticles, gold or other metal particles, lipid particles, liposomes, viral transduction, viral particles, cell-cell fusion, ballistics, microinjection, and exosome intake.

In some embodiments, the GEN-BE27 variant fusion protein comprises a nuclear localization signal (NLS), e.g., an SV40 NLS, to direct the RNP to enter a nucleus. In some embodiments, the protein (e.g., GEN-BE27 variant fusion) comprises an importin beta binding (IBB) domain sequence, e.g., to promote import of the polypeptide into a cell nucleus, e.g., by an importin (see, e.g., Lott and Cingolani (2011), Biochim Biophys Acta 1813(9): 1578-92, incorporated herein by reference).

In some embodiments, an RNA is introduced into a cell that expresses a GEN-BE27 variant fusion. In some embodiments, crRNA/tracrRNA complexes (e.g., comprising a crRNA and/or a trarcrRNA) are introduced into cells stably expressing a GEN-BE27 variant fusion. In some embodiments, labeled sgRNA is introduced into cells stably expressing a GEN-BE27 variant fusion.

### Gene Editing Nucleases (GEN)

In some embodiments, the technology comprises use of a GEN-BE27 variant fusion comprising a GEN that is, e.g., a TALEN, a meganuclease, a ZFN, etc. fused to one or more BE27 variant domains. In some embodiments, a TALEN, a meganuclease, or a ZFN is fused to a plurality of BE27 variant domains (e.g., 1, 2, 3, 4, 5, 6, 7, 8, 9, 10, 11, 12, 13, 14, 15, 16, 17, 18, 19, 20, 21, 22, 23, 24, or 25 or more BE27 variant domains). In some embodiments, a polypeptide comprises a gene editing nuclease (e.g., a TALEN, meganuclease, ZFN, etc.) and a plurality of BE27 variant domains arranged serially, e.g., in a tandem array. In some embodiments, a polypeptide comprises a gene editing nuclease (e.g., a TALEN, meganuclease, ZFN, etc.) fused to a plurality of BE27 variant domains separated by one or more linker sequences (e.g., separating one or more of the plurality of the BE27 variant domains).

In some embodiments, the technology comprises compositions, methods, kits, uses, reaction mixtures, and systems comprising and/or comprising use of a TALEN-BE27 variant to facilitate and/or stabilize the assembly of RAD51 at DSB to further improve the rate of HDR. In particular, in some embodiments, the technology comprises a TALEN in which the FokI is replaced with one or more BE27 variant domains as described herein.

ZFN, TALEN, and CRISPR/Cas9 proteins and activities are efficient in generating DSBs in the genome that can lead to a functional knockout of the targeted gene or used to integrate a DNA sequence at a specific locus (K) in the genome in a number of species (see, e.g., Carlson et al. (2012) “Efficient TALEN-mediated gene knockout in livestock” Proceedings of the National Academy of Sciences of the United States of America 109(43): 17382-87; Clark et al. (2011) “A TALE of two nucleases: gene targeting for the masses?” Zebrafish 8(3): 147-49, each of which is incorporated herein by reference). Similar to production of DSB by CRISPR proteins, NHEJ and HDR function to repair DSBs produced by TALEN and ZFN. Further, in NHEJ, the break ends are directly ligated without the need for a homologous template, thus leading to generally unpredictable insertions or deletions (indels) at the targeting sites. HDR may take place, in addition to NHEJ, when homologous donor templates are present, leading to correct repair or knock-in events. ZFN and Cas9 have been used to produce KO in rabbits (see, e.g., Yang et al. (2014) “Effective gene targeting in rabbits using RNA-guided Cas9 nucleases” J Mol Cell Biol 6(1): 97-99; Yang et al. (2013) “Production of apolipoprotein C-III knockout rabbits using zinc finger nucleases” Journal of Visualized Experiments: JoVE (81): e50957, each of which is incorporated herein). The KO rates using the Cas9 system ranged from 10-100% in vitro and 32.1-83.3% in vivo (id). However, the frequency of HDR appears to be much lower than that of NHEJ. Without any intervention, the HDR/NHEJ ratio calculated by the number of indel events over that of knock-in events was below 10% in the rabbit system, consistent with reports in other species. For example, Gonzalez et al reported 2-3% HDR rates vs. 13-49% indel rates in human ES and iPS cells in 2014 (see, e.g., Zhu et al. (2014) “The iCRISPR platform for rapid genome editing in human pluripotent stem cells” Methods in enzymology 546: 215-250, incorporated herein by reference). Likewise, in one mouse study, the NHEJ-mediated gene editing was 28-50%, whereas the HDR-mediated knock-in was below 10% (Wang et al. (2013) “One-step generation of mice carrying mutations in multiple genes by CRISPR/Cas-mediated genome engineering” Cell 153(4): 910-918, incorporated herein by reference). Collectively, the HDR events take place at ⅓ or even lower rates than the NHEJ events.

Such low knock-in rate has become a bottleneck problem for the broad application of customizable nuclease systems (e.g., TALEN, Cas9, and ZFN) in some biomedical research applications, because for reliable disease modeling and gene correction it is often necessary that a specific change be introduced to the sequence. Even for gene addition therapy, it is desirable that such addition is location- and copy-number controlled, which has been demonstrated by knock-in to the ROSA26 or similar safe harbor locus. Accordingly, provided herein are TALEN-BE27 variant fusions (e.g., TALEN fused to BE27-S15A, BE27-S22Y, or BE27-S22D), methods of using TALEN-BE27 variant fusions, kits comprising TALEN-BE27 variant fusions, and systems comprising TALEN-BE27 variant fusions. Accordingly, also provided herein are ZFN-BE27 variant fusions (e.g., ZFN fused to BE27-S15A, BE27-S22Y, or BE27-S22D), methods of using ZFN-BE27 variant fusions, kits comprising ZFN-BE27 variant fusions, and systems comprising ZFN-BE27 variant fusions.

### Methods

In some embodiments, the technology provided herein relates to methods for genetically editing a target DNA (e.g., producing a knockin), e.g., in a cell (e.g., a living cell, e.g., a living primary cell). In some embodiments, a method comprises contacting a target with a GEN-BE27 variant fusion. In some embodiments, methods comprise contacting a target DNA with a RNP complex (a “targeting complex”), which complex comprises a DNA-targeting RNA and a GEN-BE27 variant fusion (e.g., a Cas9-BE27 variant fusion), and a donor nucleic acid comprising a nucleic acid to insert at the target locus. In some embodiments, the donor nucleic acid is a targeting vector and/or a single-stranded nucleic acid comprising the nucleic acid to insert at the target locus flanked by nucleic acid complementary to the target site (“homology arms”).

In some embodiments for introducing small insertions (e.g., an insert less than approximately 50 bp, e.g., (50, 45, 40, 35, 30, 25, 20, 15, 10, 5, or 2 basepairs) or a single point mutation (e.g., 1 nt or 1 bp), the technology comprises use of a single-stranded DNA (ssDNA) oligonucleotide as the donor nucleic acid (e.g., for transfection into the target cell). In some embodiments, the ssDNA oligonucleotide comprises approximately 100-150 bp of identity and/or high homology with the target site flanking the small insert or point mutation, thus providing approximately 50-75 bp to each “homology arm” flanking each side of the mutation. In some embodiments, for introducing large insertions (e.g., insertions or deletions comprising more than approximately 50 bp, approximately 100 bp, approximately 1000 bp or more), a plasmid is typically used as the donor nucleic acid. In some embodiments for introducing large insertions, two homology arms comprising approximately 300 to 800 bp flank the desired insertion or mutation. In some embodiments, the size of such a plasmid donor is approximately 5 Kbp (see, e.g., Yang et al., Cell 154(6): 1370-1379, 2013, incorporated herein by reference). In some embodiments, homology arms that are 800 bp in length are used for efficient knockin of a 1-Kbp fragment.

In some embodiments, the donor construct is introduced into the cell in the form of a linear nucleic acid or cleaved within the cell to produce a linear nucleic acid. In some embodiments, it is delivered by any method appropriate for introducing nucleic acids into a cell. For example, the donor construct can be introduced into the cell by a variety of means known in the art, including transfection, calcium phosphate-DNA co-precipitation, DEAE-dextran-mediated transfection, polybrene-mediated transfection, electroporation, microinjection, transduction, cell fusion, liposome fusion, lipofection, protoplast fusion, retroviral infection, use of a gene gun, use of a DNA vector transporter, and biolistics (e.g., particle bombardment) (See e.g., Wu et al., 1992, J. Biol. Chem., 267:963-967; Wu and Wu, 1988, J. Biol. Chem., 263:14621-14624; and Williams et al., 1991, Proc. Natl. Acad. Sci. USA 88:2726-2730, each of which is incorporated herein by reference). Receptor-mediated DNA delivery approaches can also be used (Curiel et al., 1992, Hum. Gene Ther., 3:147-154; and Wu and Wu, 1987, J. Biol. Chem., 262:4429-4432), each of which is incorporated herein by reference.

As discussed herein, a DNA-targeting RNA and a polypeptide (a GEN-BE27 variant fusion (e.g., a Cas9-BE27 variant fusion)) form a ribonucleoprotein (RNP) complex. The DNA-targeting RNA provides target specificity to the RNP complex by comprising a nucleotide sequence that is complementary to a sequence of a target DNA. The polypeptide (a GEN-BE27 variant fusion (e.g., a Cas9-BE27 variant fusion)) of the RNP complex provides the site-specific activity. In some embodiments, an RNP complex produces a DSB in a target DNA. The target DNA may be, for example, naked DNA in vitro, chromosomal DNA in cells in vitro, chromosomal DNA in cells in vivo, etc.

In some embodiments, the RNP complex produces a DSB in a target DNA at a target DNA sequence defined by the region of complementarity between the DNA-targeting RNA and the target DNA. In some embodiments, when the polypeptide is a GEN-BE27 variant fusion (e.g., a Cas9-BE27 variant fusion), site-specific nuclease activity produces DSB in the target DNA at locations determined by both (i) base-pairing complementarity between the DNA targeting RNA and the target DNA; and (ii) a short motif (referred to as the protospacer adjacent motif (PAM)) in the target DNA. In some embodiments (e.g., when Cas9 from S. pyogenes, or a closely related Cas9, is used), the PAM sequence of the non-complementary strand is 5′-XGG-3′, where X is any DNA nucleotide and X is immediately 3′ of the target sequence of the non-complementary strand of the target DNA. As such, the PAM sequence of the complementary strand is 5′-CCY-3′, where Y is any DNA nucleotide and Y is immediately 5′ of the target sequence of the complementary strand of the target DNA. In some such embodiments, X and Y can be complementary and the X-Y base pair can be any base pair (e.g., X=C and Y=G; X=G and Y=C; X=A and Y=T, X=T and Y=A). In some embodiments, the RNP has no requirement for a PAM sequence.

In some embodiments, methods comprise a step of producing a polypeptide (e.g., a GEN-BE27 variant fusion (e.g., a Cas9-BE27 variant fusion) and/or a modified variant thereof) in vitro. In some embodiments, methods comprise a step of producing a nucleic acid in vitro, e.g., an RNA, e.g., one or more of a tracrRNA, a crRNA, and/or a sgRNA. In some embodiments, methods comprise a step of folding and/or assembling RNA (e.g., folding and/or annealing a tracrRNA and a crRNA; folding a sgRNA; folding and/or annealing a dgRNA). In some embodiments, methods comprise a step of assembling an RNP complex in vitro, e.g., an RNP comprising a polypeptide (e.g., a GEN-BE27 variant fusion (e.g., a Cas9-BE27 variant fusion)) and one or more RNA molecules. In some embodiments, methods comprise a step of introducing an RNP into a cell (e.g., a living cell, e.g., a living primary cell).

In some embodiments, multiple DNA-targeting RNAs and/or multiple RNPs are used simultaneously to simultaneously modify (e.g., by knockin) different nucleic acid sequences on the same target DNA or on different target DNAs, e.g., to provide a multiplex method. In some embodiments, two or more DNA-targeting RNAs target the same gene or transcript or locus. In some embodiments, two or more DNA-targeting RNAs target different unrelated loci. In some embodiments, two or more DNA-targeting RNAs target different, but related loci.

In some embodiments, the polypeptide (e.g., a GEN-BE27 variant fusion (e.g., a Cas9-BE27 variant fusion) and/or a modified variant thereof) is provided directly as a protein. In some embodiments, a nucleic acid is introduced into a cell and the polypeptide (e.g., a GEN-BE27 variant fusion (e.g., a Cas9-BE27 variant fusion) and/or a modified variant thereof) is expressed from the nucleic acid in the cell. As one non-limiting example, fungi (e.g., yeast) can be transformed with exogenous protein, nucleic acid, and/or RNP complexes using spheroplast transformation (see Kawai et al., Bioeng Bugs. 2010 November-December; 1(6): 395-403, “Transformation of Saccharomyces cerevisiae and other fungi: methods and possible underlying mechanism”; and Tanka et al., Nature. 2004 Mar. 18; 428(6980):323-8: “Conformational variations in an infectious protein determine prion strain differences”; each of which is herein incorporated by reference). Thus, a polypeptide (e.g., a GEN-BE27 variant fusion (e.g., a Cas9-BE27 variant fusion) and/or a modified variant thereof), nucleic acid (e.g., RNA), and/or a RNP can be incorporated into a spheroplast and the spheroplast can be used to introduce the RNP into a yeast cell. An RNP can be introduced into a cell (provided to the cell) by any convenient method; such methods are known to those of ordinary skill in the art. As another non-limiting example, an RNP can be injected directly into a cell, e.g., a human cell, a cell of a zebrafish embryo, the pronucleus of a fertilized mouse oocyte, etc.

In some embodiments, methods comprise introducing into a target cell (e.g., a eukaryotic cell) one or more nucleic acids (e.g., a subject donor DNA molecule, a nucleic acid comprising nucleotide sequences encoding a GEN-BE27 variant fusion, etc.). Methods of introducing a nucleic acid into a cell are known in the art and any convenient method can be used (e.g., electroporation, lipofection, nucleofection, injection, viral vectors, etc.). In some embodiments, a DNA molecule is introduced into a cell in a composition that also includes a GEN-BE27 variant fusion.

When one or more nucleic acids are used that include nucleotides encoding a GEN-BE27 variant fusion, the sequence encoding the GEN-BE27 variant fusion can be codon-optimized. A sequence encoding any suitable GEN-BE27 variant fusion can be codon optimized. As a non-limiting example, if the intended host cell were a mouse cell, then a mouse codon-optimized nucleotide sequence encoding a GEN-BE27 variant fusion (or variant thereof) would be suitable. While codon optimization is not required, it is acceptable and may be preferable in certain cases.

In some embodiments, one or more of the above nucleic acids is provided in a recombinant expression vector. In some embodiments, the recombinant expression vector is a viral construct, e.g., a recombinant adeno-associated virus construct (see, e.g., U.S. Pat. No. 7,078,387, incorporated herein by reference), a recombinant adenoviral construct, a recombinant lentiviral construct, a recombinant retroviral construct, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984; and WO 95/00655, each of which is incorporated herein by reference); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617, each of which is incorporated herein by reference); SV40; herpes simplex virus; human immunodeficiency virus (see, e.g., Miyoshi et al., PNAS 94:10319 23, 1997; Takahashi et al., J Virol 73:7812 7816, 1999, each of which is incorporated herein by reference); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example, e.g., for eukaryotic host cells: pXT1, pSG5 (Stratagene), pSVK3, pBPV, pMSG, and pSVLSV40 (Pharmacia). However, any other vector may be used if it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544, incorporated herein by reference).

In some embodiments, a nucleotide sequence encoding a GEN-BE27 variant fusion is operably linked to a control element, e.g., a transcriptional control element, such as a promoter. The transcriptional control element may be functional in either a eukaryotic cell (e.g., a mammalian cell) and/or a prokaryotic cell (e.g., a bacterial or an archaeal cell), e.g., in cases where a GEN-BE27 variant fusion protein will be isolated/purified prior to the contacting step. In some embodiments, a nucleotide sequence encoding a GEN-BE27 variant fusion protein is operably linked to multiple control elements that allow expression of the nucleotide sequence encoding a GEN-BE27 variant fusion protein in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is within the level of ordinary skill in the art. In some embodiments, a promoter is chosen to achieve a desirable level expression (e.g., which, in some embodiments, is as high as possible and, in some embodiments, is above or below a desired threshold, e.g., to achieve the desired goal while reducing off-target effects). The expression vector may also contain a ribosome binding site for translation initiation and a transcription terminator. The expression vector may also include appropriate sequences for amplifying expression. The expression vector may also include nucleotide sequences encoding one or more protein tag(s) (e.g., a 6×His tag, hemagglutinin tag, green fluorescent protein, etc.) that is/are fused to a GEN-BE27 variant fusion protein, thus resulting in one nor more chimeric polypeptides.

In some embodiments, a nucleotide sequence encoding a GEN-BE27 variant fusion protein is operably linked to an inducible promoter. In some embodiments, a nucleotide sequence encoding a GEN-BE27 variant fusion protein is operably linked to a constitutive promoter.

Methods of introducing a nucleic acid into a host cell are known in the art, and any known method can be used to introduce a nucleic acid (e.g., an expression construct) into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012, incorporated herein by reference), and the like.

### Cells

In some embodiments of the technology provided herein, the technology finds use to modify (e.g., by knockin) nucleic acids in mitotic or post-mitotic cells in vivo and/or ex vivo and/or in vitro. Because the DNA-targeting RNA provides specificity by hybridizing to target DNA, a mitotic and/or post-mitotic cell of interest may include a cell from any organism (e.g., a eukaryotic cell, a bacterial cell, an archaeal cell, a cell of a single-cell eukaryotic organism, a plant cell, an algal cell, e.g., Botryococcus braunii, Chlamydomonas reinhardti, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.).

Any type of cell may be of interest (e.g., a stem cell (e.g., an embryonic stem (ES) cell, an induced pluripotent stem (iPS) cell, a germ cell); a somatic cell (e.g., a fibroblast, a hematopoietic cell, a neuron, a muscle cell, a bone cell, a hepatocyte, a pancreatic cell); an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”. “primary cell lines”, and “primary cultures” are used interchangeably herein to refer to cells and cells cultures that have been derived from a subject and allowed to grow in vitro for a limited number of passages (e.g., “splittings”) of the culture. For example, primary cultures are cultures that may have been passaged 0 times, 1 time, 2 times, 4 times, 5 times, 10 times, or 15 times, but not enough times to go through the crisis stage. Typically, the primary cell lines of the present technology are maintained for fewer than 10 passages in vitro. Target cells are in many embodiments unicellular organisms or are grown in culture.

In some embodiments, primary cells are obtained from an individual by any convenient method. For example, leukocytes may be conveniently obtained by apheresis, leukocytapheresis, density gradient separation, etc., while cells from tissues such as skin, muscle, bone marrow, spleen, liver, pancreas, lung, intestine, stomach, etc. are most conveniently obtained by biopsy. An appropriate solution may be used for dispersion or suspension of the obtained cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

### Samples

In some embodiments, nucleic acids (e.g., DNA or RNA, e.g., chromosomes, genes, genetic loci, genetic markers, etc.) are obtained, derived, and/or isolated from a biological sample containing a variety of other components, such as proteins, lipids, and non-target nucleic acids. In some embodiments, samples are obtained from and/or comprise and/or are derived or prepared from a variety of materials (e.g., cellular material (live or dead), extracellular material, viral material, environmental samples (e.g., metagenomic samples), synthetic material (e.g., amplicons such as provided by PCR or other temperature-cycled or isothermal amplification technologies)), obtained from an animal, plant, bacterium, archaeon, fungus, or any other organism. Biological samples for use in the present technology include viral particles or preparations thereof. In some embodiments, samples are obtained directly from an organism or from a biological sample obtained from an organism, e.g., blood, urine, cerebrospinal fluid, seminal fluid, saliva, sputum, stool, hair, sweat, tears, skin, amniotic fluid, and tissue (e.g., umbilical tissue). Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a sample or a source of a sample for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. In particular embodiments, the sample comprises cultured cells, such as a primary cell culture or a cell line. In some specific embodiments, the sample comprises live primary cells.

In some embodiments, samples (e.g., cells or tissues) are infected with a virus or other intracellular pathogen. A sample can also be isolated from a non-cellular origin, e.g. amplified/isolated nucleic acid (e.g., in some embodiments, that has been stored in a freezer).

In some embodiments of the technology, the technology is applied in vivo, ex vivo, and/or in vitro. In some embodiments, the technology is used on a sample in situ, e.g., without removing it from a subject or a patient. In some embodiments, the sample is a crude sample, a minimally treated cell lysate, or a biofluid lysate.

### Kits

In some embodiments, the technology provides a kit for modifying a nucleic acid (e.g., producing a knockin in a nucleic acid). In some embodiments, a kit comprises a GEN-BE27 variant fusion (e.g., a GEN-BE27 variant fusion (e.g., a Cas9-BE27 variant fusion) and/or a modified variant thereof). In some embodiments, a kit comprises a GEN-BE27 variant fusion comprising a TALEN, ZFN, and/or meganuclease fused to one or more BE27 variant domains (e.g., BE27-S15A, BE27-S22Y, or BE27-S22D).

In some embodiments, a kit comprises: a) a DNA-targeting RNA or a nucleic acid comprising a nucleotide sequence encoding a DNA-targeting RNA, wherein the DNA-targeting RNA comprises: i) a first segment comprising a nucleotide sequence that is complementary to a target sequence in the target DNA; and ii) a second segment that interacts with a polypeptide to form an RNP as described herein; and, optionally, b) a buffer. In some embodiments, a kit further includes one or more additional reagents, where such additional reagents can be selected from: a buffer; a wash buffer; a control reagent; a control expression vector or RNA polynucleotide; a reagent for in vitro production of the GEN-BE27 variant fusion polypeptide from DNA; and the like. In some embodiments, the fusion protein further comprises a domain providing enhanced or improved localization (e.g., transport) to the nucleus (e.g., an NLS, an IBB, etc.) In some embodiments, components of the kit are in separate containers; in some embodiments, one or more components of a kit are combined in a single container. Further, in some embodiments, a kit can further include instructions for using the components of the kit to practice a method described herein. In some embodiments, kits comprise one or more compositions as described herein, e.g., packaged in one or more containers for use by a user. Further, in some embodiments, a kit can further include instructions for using the components of the kit to practice a method described herein.

The present disclosure provides kits for carrying out a method as described herein. In some embodiments, a kit comprises a GEN-BE27 variant fusion protein and/or a nucleic acid having a nucleotide sequence encoding a GEN-BE27 variant fusion protein. In some embodiments, a kit further comprises a linear DNA molecule (e.g., a donor molecule). In some embodiments, a kit can further include one or more additional reagents, where such additional reagents can be selected from, e.g., a dilution buffer; a reconstitution solution; a wash buffer; a control reagent; a control expression vector or RNA polynucleotide; a reagent for in vitro production of a GEN-BE27 variant fusion protein from DNA, and the like. The components of a subject kit can be in the same or different containers (in any desired combination).

In addition to embodiments comprising one or more of the above-mentioned components, a kit can further include instructions for using the components of the kit to practice a method as described herein. The instructions for practicing methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or sub-packaging), etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Systems

Some embodiments of the technology provide systems for modifying a nucleic acid (e.g., producing a knockin). Systems according to the technology comprise, e.g., polypeptides (e.g., a GEN-BE27 variant fusion (e.g., a Cas9-BE27 variant fusion) and/or a modified variant thereof). In some embodiments, systems comprise RNAs (e.g., dgRNA, sgRNA). Related embodiments provide expression systems (e.g., comprising nucleic acids encoding the polypeptides and/or RNAs; and one or more expression hosts) for producing polypeptides and/or RNAs described herein using an in vitro system. In some embodiments, the systems further comprise an in-vitro system for assembly of RNP complexes. Some embodiments comprise fluid handling (e.g., in some embodiments, microfluidics) components for transporting samples, reagents, and other compositions for modifying a nucleic acid with an RNP. Some embodiments comprise components for fluid storage and fluid waste storage. In some embodiments, one or more components is/are provided to the system in the form of a kit.

In some embodiments, systems comprise a cell (e.g., a cultured cell, a primary cell, e.g., a cell in a sample obtained from a subject). For example, in some embodiments, systems comprise a cell comprising a GEN-BE27 variant fusion as described herein. In particular embodiments, systems comprise a cell, a polypeptide, and one or more RNA molecules. Some embodiments comprise a computer and software encoding instructions for the computer to perform. For instance, some embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data.

Some embodiments comprise a storage medium and memory components. Memory components (e.g., volatile and/or nonvolatile memory) find use in storing instructions (e.g., an embodiment of a process as provided herein) and/or data. Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship. For example, some embodiments provide systems in which a processor is remote from one or more other components of the system, e.g., to provide a system arranged in a cloud computing arrangement.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, Swift, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, macOS, NeXTSTEP, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

In accordance with such a computer system, some embodiments of the technology provided herein further comprise functionalities for collecting, storing, and/or analyzing data. For example, some embodiments contemplate a system that comprises a processor, a memory, and/or a database for, e.g., storing and executing instructions, analyzing data, performing calculations using the data, transforming the data, and storing the data. In some embodiments, an algorithm applies a statistical model to the data.

Many diagnostics involve determining the presence of, absence of, identity of, or a nucleotide sequence of, one or more nucleic acids. Thus, in some embodiments, an equation comprising variables representing the presence, absence, identity, concentration, amount, or sequence properties of multiple nucleic acids produces a value that finds use in making a diagnosis or assessing the presence or qualities of a nucleic acid. As such, in some embodiments this value is presented by a device, e.g., by an indicator related to the result (e.g., an LED, an icon on a display, a sound, or the like). In some embodiments, a device stores the value, transmits the value, or uses the value for additional calculations.

Thus, in some embodiments, the present technology provides the further benefit that a clinician, who is not likely to be trained in genetics or molecular biology, need not understand the raw data. The data are presented directly to the clinician in its most useful form. The clinician is then able to utilize the information to optimize the care of a subject. The present invention contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed and a profile is produced that is specific for the diagnostic or prognostic information desired for the subject. The profile data are then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may choose further intervention or counseling based on the results. In some embodiments, the data are used for research use. For example, the data may be used to further optimize the inclusion or elimination of markers as useful indicators of a particular condition associated with the disease.

### Uses

In some embodiments, compositions, kits, and methods find use for the integration of a donor DNA molecule into any desirable nucleic acid, e.g., a supercoiled target DNA molecule, a chromosome, an extrachromosomal element, etc. The following uses are merely illustrative examples and are by no means meant to limit the use of the subject methods. In some embodiments, the compositions, kits, and methods find use for in vitro use outside of a cell (e.g., to modify a plasmid DNA, to modify an isolated chromosomal DNA, etc.). In some embodiments, the compositions, kits, and methods find use inside of a eukaryotic cell (e.g., in vitro and/or in in vivo and/or ex vivo). In some embodiments, the compositions, kits, and methods are used to insert and/or modify a control element (e.g., a transcriptional control element such as an enhancer, a promoter, a transcription terminator, etc.). In some embodiments, the compositions, kits, and methods find use to modify a target gene (e.g., in some cases disrupting the expression of the target gene, in some cases, modifying the transcribed RNA, etc.). In some embodiments, the compositions, kits, and methods find use to modify a coding and/or a non-coding sequence (e.g., modify a gene coding sequence, modify a sequence that codes for a non-coding RNA such as a microRNA).

In some embodiments, The technologies described herein find use in, e.g., research, imaging, diagnostics, and treatment of patients. Applications include research applications; diagnostic applications; industrial applications; and treatment applications. Research applications include, e.g., characterizing, detecting, modifying, and/or identifying nucleic acids in a cell (e.g., a living cell). Further uses of embodiments of the technology described herein include one or more of the following: genome imaging; copy number analysis; analysis of living cells; detection of highly repetitive genome sequence or structure; detection of complex genome sequences or structures; detection of gene duplication or rearrangement; chromosomal labeling; large scale diagnostics of diseases and genetic disorders related to genome deletion, duplication, and rearrangement; use of multiple unique sgRNAs for high-throughput imaging and/or diagnostics; multicolor differential detection of target sequences; identification or diagnosis of diseases of unknown cause or origin; and 4-dimensional (e.g., time-lapse) or 5-dimensional (e.g., multicolor time-lapse) imaging of cells (e.g., live cells), tissues, or organisms.

### Uses of a GEN-BE27 Variant Fusion to Modify a Cell or Organism

The technology in some embodiments comprises a method of modifying a cell or organism. The cell may be a prokaryotic cell or a eukaryotic cell. The cell may be a mammalian cell. The mammalian cell many be a non-human primate, bovine, porcine, rodent, or mouse cell. The cell may be a non-mammalian eukaryotic cell such as poultry, fish, or shrimp. The cell may also be a plant cell. The plant cell may be of a crop plant such as cassava, corn, sorghum, wheat, or rice. The plant cell may also be a cell of an algae, tree, or vegetable. The modification introduced to the cell by the present technology may be such that the cell and progeny of the cell are altered for improved production of biologic products such as an antibody, starch, alcohol, or other desired cellular output. The modification introduced to the cell by the present technology may be such that the cell and progeny of the cell include an alteration that changes the biologic product produced.

The technology may comprise use of one or more different vectors. In some embodiments of the technology, the GEN-BE27 variant fusion protein is codon optimized for expression the desired cell type, preferentially a eukaryotic cell, preferably a mammalian cell or a human cell.

In some embodiments, packaging cells are used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, hut lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, one or more vectors described herein are used to produce a non-human transgenic animal or transgenic plant. In some embodiments, the transgenic animal is a mammal, such as a mouse, rat, or rabbit. Methods for producing transgenic animals and plants are known in the art, and generally begin with a method of cell transfection, such as described herein. In another embodiment, a fluid delivery device with an array of needles (see, e.g., US Patent Publication No. 20110230839, incorporated herein by reference) may be contemplated for delivery of a GEN-BE27 variant fusion to solid tissue. A device of US Patent Publication No. 20110230839 for delivery of a fluid to a solid tissue may comprise a plurality of needles arranged in an array; a plurality of reservoirs, each in fluid communication with a respective one of the plurality of needles; and a plurality of actuators operatively coupled to respective ones of the plurality of reservoirs and configured to control a fluid pressure within the reservoir.

In some embodiments, the technology provides for methods of modifying a target polynucleotide in a eukaryotic cell. In some embodiments, the method comprises allowing a nucleic acid-targeting complex to bind to the target polynucleotide to effect cleavage of said target polynucleotide thereby modifying the target polynucleotide, wherein the nucleic acid-targeting complex comprises a nucleic acid-targeting effector protein complexed with a guide RNA hybridized to a target sequence within said target polynucleotide.

In some embodiments, the technology provides a method of modifying expression of a polynucleotide in a eukaryotic cell. In some embodiments, the method comprises allowing a nucleic acid-targeting complex to bind to the polynucleotide such that said binding results in increased or decreased expression of said polynucleotide; wherein the nucleic acid-targeting complex comprises a nucleic acid-targeting effector protein complexed with a guide RNA hybridized to a target sequence within said polynucleotide.

In some embodiments, the technology provides a method of inserting a polynucleotide into the genome of a eukaryotic cell. In some embodiments, the method comprises allowing a nucleic acid-targeting complex to bind to the polynucleotide such that said binding results in the insertion of said polynucleotide at a target locus; wherein the nucleic acid-targeting complex comprises a nucleic acid-targeting effector protein complexed with a guide RNA hybridized to a target sequence within said polynucleotide.

Although the disclosure herein refers to certain illustrated embodiments, it is to be understood that these embodiments are presented by way of example and not by way of limitation.

### EXAMPLES

Provided herein is a modified CRISPR/Cas9 technology for improved gene editing. During the development of embodiments of the technology provided herein, experiments indicated that the improved CRISPR technology increased the KI efficiency of a large-size gene knock-in. Furthermore, the improved CRISPR technology provided herein reduced off-target insertion and deletion (“indel”) events at several genetic loci (e.g., VEGFA, EMX1, and FNACF) known to be especially prone to off-target CRISPR modification. The CRISPR technology provided herein finds use in a range of gene editing research and therapeutics applications.

Conventional CRISPR/Cas9 technologies create double stranded breaks (DSBs) at a target locus in a genome. Two major mechanisms, non-homologous end joining (NHEJ) and homology directed repair (HDR), function to repair DSBs. The majority of DSBs are repaired by NHEJ mechanisms (see, e.g., Zhu et al. (2014) “The iCRISPR platform for rapid genome editing in human pluripotent stem cells” Methods in enzymology 546: 215-250; Wang et al. (2013) “One-step generation of mice carrying mutations in multiple genes by CRISPR/Cas-mediated genome engineering” Cell 153(4): 910-918) NHEJ directly ligates break ends without using a homologous template, which produces insertions and deletions (indels) in a genome with low predictability or insufficient predictability to provide a robust and useful gene editing tool. In contrast, HDR occurs when homologous donor templates are present, thus producing correct repair or knock-in events. Accordingly, when DSBs are repaired by the HDR pathway in the presence of a donor template comprising a nucleic acid insert, knock-in of the insert takes place with increased specificity for the target locus. In particular, the use of a pre-assembled ribonucleoprotein (RNP), comprising a CRISPR/Cas9 protein and a guide RNA (gRNA), and a short single-stranded oligodeoxynucleotide (ssODN) donor template induces the single-strand annealing (SSA) pathway for nucleic acid repair, which has been shown to improve the precision of producing mutations to efficiencies in the double digits. Despite these advances, use of CRISPR technologies to knock in a large-fragment donor template by homologous recombination (HR) pathways has achieved efficiencies of no greater than approximately 1%.

Fusion of BE27 to genome editing nucleases (GEN) (e.g., Streptococcus pyogenes Cas9 (spCas9)) improves the efficacy and safety profiles of the recipient GEN. See, e.g., Int'l pat. app. No. PCT/US2019/030913, incorporated herein by reference. During the development of embodiments of the technology provided herein, amino acids in the BE27 polypeptide were identified that modulate homology directed repair (HDR). These amino acids include serine at position 15 (S15) and the serine at position 22 (S22).

During the development of embodiments of the technology described herein, new Cas9 variants (referred to by the terms “Cas9-A”, “Cas9-Y”, and “Cas9-D”; collectively referred to by the term “miCas9-A/Y/D”) were constructed by fusing spCas9 with a BE27 variant comprising a nucleic acid mutation encoding the amino acids substitutions S15A, S22Y, or S22D in the BE27 amino acid sequence (referred to by the terms “BE27-S15A”, “BE27-S22Y”, and BE27-S22D; collectively referred to by the term “BE27-A/Y/D”).

During the development of embodiments of the technology described herein, data collected in experiments using Cas9-A/Y/D indicated that on-target and off-target insertion and deletion (indel) events occurred significantly less frequently without decreasing the efficiency of precise gene editing. Further, use of Cas9-A/Y/D increased the rates of large size gene knock-in. The data indicated that the BE27-S15A, BE27-S22Y, and BE27-S22D variants provide a universal polypeptide that can be fused with any GEN having an activity that produces double-stranded breaks (DSBs), e.g., to generate a GEN-BE27-A/Y/D fusion protein with increased efficacy and safety profiles in genome editing applications.

### Materials and Methods

Human fibroblast cells (Cat #CRL2522) were acquired from American Type Culture Collection (ATCC, Manassas, VA). Cells were cultured with RPMI 1640 Medium (Cat #11875119, ThermoFisher Scientific, Waltham, MA) supplemented with 10% fetal bovine serum (FBS, Cat #FBS1824-001, Nucleus Biologics, San Diego, CA). Human Ad293 cells (Cat #240085, Agilent, Santa Clara, CA) were cultured in Dulbecco's Modified Eagle Medium (DMEM) (Cat #11995-065, ThermoFisher Scientific) supplemented with 10% fetal bovine serum. Oligonucleotides were commercially synthesized by IDT.

As described in Int'l pat. app. No. PCT/US2019/030913, incorporated herein by reference, BE27 is a 36-amino acid peptide having an amino acid sequence according to SEQ ID NO: 1. As described previously in PCT/US2019/030913, incorporated herein by reference, Cas9-BE27 protein fusions were produced that comprise a dead-spCas9, a linker sequence, and BE27. Cas9-BE27 variants described herein were constructed by replacing the BE27 of the Cas9-BE27 fusions with BE27-S15A, BE27-S22Y, or BE27-S22D to produce Cas9-A, Cas9-Y, and Cas9-D, respectively.

sgRNA-expressing plasmids were produced by ligating annealed DNA oligonucleotide duplexes into phU6-sgRNA (Cat #53188, Addgene). The sgRNA sequences are provided below:

| Guide RNA name | Target gene | Guide RNA sequence | SEQ ID NO |
|---|---|---|---|
| sg-EMX1 | EMX1 | GAGTTAGAGCAGAAGAAGAAAGG | SEQ ID NO: 2 |
| sg-VEGFA3 | VEGFA | GGTGAGTGAGTGTGTGCGTGTGG | SEQ ID NO: 3 |
| sg-FANCF2 | FANCF2 | GCTGCAGAAGGGATTCCATGAGG | SEQ ID NO: 4 |

For RNP experiments, sgRNAs were transcribed in vitro using a gRNA synthesis kit (Cat #A29377, ThermoFisher Scientific).

Knock-in donor templates were constructed using a GFP coding sequence and target sequences. In particular, double-stranded DNA donor templates were constructed using a GFP coding sequence flanked by left and right homologous arms corresponding to target sites. 5′-biotin modified primers were used for PCR to produce biotinylated GFP expressing donor templates in the experiments. The double-stranded knock-in donor nucleic acid named “GFP-donor-1k-AAVS1” was produced to target the AAVS1 gene using a guide RNA (“sg-AAVS1”) having a sequence GGGGCCACTAGGGACAGGATTGG (SEQ ID NO: 5). The left homology arm had a length of 804 bp, the right homology arm had a length of 837 bp, and the knock-in fragment had a length of 989 bp. 5′-biotin modified primers were used for PCR to produce biotinylated GFP expressing donor templates in the experiments.

Electroporation was performed using a tube electroporation machine (Model #CTX-1500A LE, Celetrix, Manassas, VA) to deliver Cas9 elements (e.g., Cas9 plasmid DNA or RNP, gRNAs, and donor templates) to cells. 2-3×10⁶ cells were resuspended in 120 μL of electroporation buffer (Cat #13-0104, Celetrix). To deliver Cas9 in plasmid DNA form, 4.5 μg of Cas9-expressing plasmid DNA and 1.5 μg of sgRNA-expressing plasmid DNA were added to the buffer. To deliver Cas9 in RNP form, 10 μg of Cas9 was pre-mixed with 3.3 μg of gRNA and added to the buffer. For both delivery methods, 4 μg of GFP-expressing donor templates or 10 μg ssODN donor templates were added to the buffer for knock-in experiments. The electroporation conditions were 620 V for 30 ms for fibroblast cells, airway epithelial cells iPSCs, and Ad293 cells. The electroporation conditions were 635 V for 30 ms for HSCs.

T7EI (T7 endonuclease I) assays were conducted as previously described (see, e.g., X. Xu, “Efficient homology-directed gene editing by CRISPR/Cas9 in human stem and primary cells using tube electroporation” Sci Rep 8: 11649 (2018), incorporated herein by reference). Non-perfectly matched DNA (e.g., indel sites) are recognized and cleaved by T7EI to produce two cleaved bands upon electrophoresis and visualization. Perfectly (e.g., essentially and/or substantially perfectly) matched DNA is not recognized and cleaved by T7EI, thus producing one band (the wild-type band) upon electrophoresis and visualization. The ratio of the intensity of the average of the two cleaved bands relative to the intensity of the average of the wild-type band was used as a quantitative estimate for indel efficiency.

For deep sequencing (deep-seq) and analysis, cells were harvested 48 hours after transfection and genomic DNAs were extracted with the Wizard Genomic DNA Purification Kit (Cat #A1120, Promega, Madison, WI). Targeted regions were amplified by PCR using high-fidelity PCR master mix (Cat #F532L, ThermoFisher Scientific) and oligonucleotide primers comprising the following nucleotide sequences:

| Name | Sequence | SEQ ID NO: |
|---|---|---|
| AAVS1-F | ATGTGGCTCTGGTTCTGGGT | SEQ ID |
| | | NO: 13 |
| AAVS1-R | GGAAGGAGGAGGCCTAAGGA | SEQ ID |
| | | NO: 14 |
| F2-F | TCCCAGGTGCTGACGTAGGT | SEQ ID |
| | | NO: 15 |
| F2-R | GAGAGTCGCCGTCTCCAAGG | SEQ ID |
| | | NO: 16 |
| VEGFA1-F | AGCCCATTCCCTCTTTAGCC | SEQ ID |
| | | NO: 17 |
| VEGFA1-R | GGAGTGACCCCTGGCCTT | SEQ ID |
| | | NO: 18 |
| EMX1-OT1-F | GTGGGGAGATTTGCATCTGTGGAGG | SEQ ID |
| | | NO: 19 |
| EMX1-OT1-R | GCTTTTATACCATCTTGGGGTTACAG | SEQ ID |
| | | NO: 20 |

The AAVS1-F and AAVS1-R primers were used to produce the on-target amplicon at the sg-AAVS1 target locus. The F2-F and F2-R primers were used to produce the on-target amplicon at the sg-FANCF2 target locus. The VEGFA1-F and VEGFA1-R primers were used to produce the on-target amplicon at the sg1-VEGFA target locus. The EMX-OT1-F and EMX-OT1-R primers were used to produce the amplicon at one of the sg-EMX1 off-target loci.

Products were isolated using Qiaquick gel purification kit (Cat #28706, Qiagen, Germantown, MD) and sequenced at the CCIB DNA Core Facility at Massachusetts General Hospital (Cambridge, MA). Briefly, Illumina compatible adapters comprising unique barcodes were ligated onto each sample during library construction. Libraries were pooled in equimolar concentrations for multiplexed sequencing on an Illumina MiSeq platform with 2×150 run parameters. Upon completion of the sequencing run, data were analyzed, demultiplexed, and subsequently input into an automated de novo pipeline. Analysis was completed through the MGH CCIB de novo assembler UltraCycler v1.0 and manually inspected for quality control.

A brief description of the algorithm is available at dnacore.mgh.harvard.edu/new-cgi-bin/site/pages/crispr_sequencing_pages/crispr_sequencing_algorithm.jsp.

PGE and indel rates were determined by the CRISPResso2 tool (crispresso.pinellolab.partners.org/) using .fastq files. See, e.g., K. Clement, “CRISPResso2 provides accurate and rapid genome editing sequence analysis” Nat Biotechnol 37, 224-226 (2019), incorporated herein by reference).

For analysis of the PGE and indel rates at CCR5-del32, where the primer and amplicon sequences are highly homologous with those at CCR2, the .seq files were analyzed by Blastn (blast.ncbi.nlm.nih.gov/) against the HDR amplicon sequence and the wild-type amplicon sequence.

Knockin events were analyzed by fluorescence-associated cell sorting (FACS). Cultured cells were disassociated three days post-transfection and suspended in 300 μL PBS comprising 2% FBS and filtered with a 70-μm nylon strainer. Cells were subjected to flow cytometry using the MoFlo Astrios EQs Sorter (Model #B52102, Beckman Coulter, Indianapolis, IN) at the University of Michigan Flow Cytometry Core. The data were analyzed using FlowJo (Version 10, Tree Star, Ashland, OR). The percentages of GFP positive cells were used as the indicator of gene knock-in events.

Data are presented as mean±SEM. Measurements were taken from 3 distinct samples, except for the off-target analysis on Guide-seq predicted loci where measurements were taken from 2-3 distinct samples. Unpaired t test (two-tailed) was used to compare data using GraphPad Prism 8 software (GraphPad Software, Inc., San Diego, CA). Exact P values are labeled in figures.

### Example 1—CRISPR System Design

The HDR process involves BRCA2 delivering RAD51 to a resected DNA overhang at a DSB to form a RAD51/DNA filament. With respect to the role of BRCA2, data have indicated that a RAD51 binding sequence encoded by BRCA2 exon 27 (herein referred to by the term “BE27”) guides and places RAD51 at the DSB. Construction of fusions comprising an N-terminal domain comprising Cas9 and a C-terminal domain comprising BE27 are described in Int'l pat. app. No. PCT/US2019/030913, incorporated herein by reference. In subsequent experiments, data were collected that identified two particular amino acids in the BE27 polypeptide that are important for HDR. These amino acids are a serine at amino acid 15 (S15) and a serine at amino acid 22 (S22). These amino acids are indicated in the BE27 amino acid sequence by underlining in the BE27 amino acid sequence below:

BE27
(SEQ ID NO: 1)
A L D F L S R L P L P P P V S P I C T F V S P A A Q K A F Q P P R S C G
1 5 10 15 20 25 30 36

Experiments were conducted that identified specific amino acid substitutions at these two amino acids that produced improved HDR outcomes in gene editing using fusions of Cas9 and the substituted BE27 polypeptides (Cas9-BE27 variant fusion proteins) constructed during the development of embodiments of the technology described herein. In particular, experiments were conducted during the development of embodiments of the technology described herein to produce fusions of BE27 variants (e.g., BE27-S15A, BE27-S22Y, or BE27-S22D) and Cas9 (e.g., Streptococcus pyogenes Cas9 (spCas9)). The amino acid sequences of the BE27 variants are provided below:

BE27-S15A
(SEQ ID NO: 10)
A L D F L S R L P L P P P V A P I C T F V S P A A Q K A F Q P P R S C G
1 5 10 15 20 25 30 36
BE27-S22Y
(SEQ ID NO: 11)
A L D F L S R L P L P P P V S P I C T F V Y P A A Q K A F Q P P R S C G
1 5 10 15 20 25 30 36
BE27-S22D
(SEQ ID NO: 12)
A L D F L S R L P L P P P V S P I C T F V D P A A Q K A F Q P P R S C G
1 5 10 15 20 25 30 36

Cas9 fusions were constructed using the BE27 variants to produce the Cas9-BE27 variant fusions miCas9-A, miCas9-Y, and miCas9-D. The miCas9-A, miCas9-Y, and miCas9-D fusions comprise an N-terminal domain that is the Cas9 polypeptide and a C-terminal domain that is the BE27 variant polypeptide. Data collected during these experiments indicated that the miCas9-A, miCas9-Y, and miCas9-D fusions provided an efficient deposition of RAD51 at the DSB, thus increasing the amount and/or concentration of RAD51 the DSB and, consequently, improving the HDR-mediated knock-in of large fragment inserts at the target locus.

Embodiments of the technology thus provide miCas9-A, miCas9-Y, and miCas9-D fusion proteins (collectively, miCas9 variants). In some embodiments, a miCas9 variant comprises a nuclear localization signal between the N-terminal Cas9 polypeptide and the C-terminal BE27 variant polypeptide.

In some embodiments, the technology comprises, e.g., a nucleic acid encoding a miCas9 variant (e.g., a miCas9-A, miCas9-Y, or miCas9-D), a vector comprising a nucleic acid encoding a miCas9 variant (e.g., a miCas9-A, miCas9-Y, or miCas9-D), a cell comprising and/or expressing a nucleic acid encoding a miCas9 variant (e.g., a miCas9-A, miCas9-Y, or miCas9-D), and/or a cell comprising and/or expressing a miCas9 variant (e.g., a miCas9-A, miCas9-Y, or miCas9-D). In some embodiments, the technology comprises, e.g., a nucleic acid encoding a miCas9 variant (e.g., a miCas9-A, miCas9-Y, or miCas9-D) comprising an NLS, a vector comprising a nucleic acid encoding a miCas9 variant (e.g., a miCas9-A, miCas9-Y, or miCas9-D) comprising an NLS, a cell comprising and/or expressing a nucleic acid encoding a miCas9 variant (e.g., a miCas9-A, miCas9-Y, or miCas9-D) comprising an NLS, and/or a cell comprising and/or expressing a miCas9 variant (e.g., a miCas9-A, miCas9-Y, or miCas9-D) comprising an NLS. As used herein, the term “miCas9 variant” refers both to a Cas9-BE27 variant fusion protein and a Cas9-BE27 variant fusion protein comprising an NLS. Further, as used herein, the term “miCas9” is used to refer to embodiments of the Cas9-BE27 variant fusion protein and a Cas9-BE27 variant fusion protein comprising an NLS described herein and the term “miCRISPR” is used to refer to CRISPR technologies using the Cas9-BE27 variant fusion protein and a Cas9-BE27 variant fusion protein comprising an NLS as described herein.

### Example 2—Cas9-BE27 Variant Fusion Proteins Reduce Off-Target Indel Rates

During the development of embodiments of the technology provided herein, experiments were conducted to evaluate the off-target indel rates produced at the predicted off-target site 1 using the Cas9-BE27 variant fusion proteins and the sg-EMX1 guide RNA by T7E1 assay. Results are shown in FIG. 1. The lane marked “WT” is a positive control marker for the size of the WT band expected by the T7E1 assay.

As indicated by the data provided in FIG. 1, spCas9 induced substantial off-target edits, as indicated by multiple indel bands observed below the wild-type (WT) band (FIG. 1, lane labeled “spCas9”). The Cas9-BE27 fusion previously described effectively reduced the indel rates, as indicated by the lower intensities observed for the indel bands below the WT bands (FIG. 1, lane labeled “miCas9”). A Cas9 fused to BE27 comprising a S22E substitution (miCas9-S22E) increased the intensities of the indel bands, suggesting that the S22E substitution in BE27 does not reduce off-target indel rates (FIG. 1, lane labeled “miCas9-S22E”). However, the other Cas9-BE27 variants miCas9-Y, miCas9-A and miCas9-D significantly reduced the intensities of the indel bands. In particular, the intensities of the indel bands for miCas9-Y, miCas9-A and miCas9-D were much lower than the intensities of the indel bands for spCas9 and the Cas9-BE27 fusion (FIG. 1, lanes labeled “miCas9-Y”, “miCas9-A”, and “miCas9-D”, respectively). In fact, indel bands were undetectable for miCas9-Y, miCas9-A, and miCas9-D by T71 assay, indicating a near 100% reduction of the off-target indel edits.

### Example 3—Cas9-BE27 Variant Fusion Proteins Reduce On-Target Indel Rates

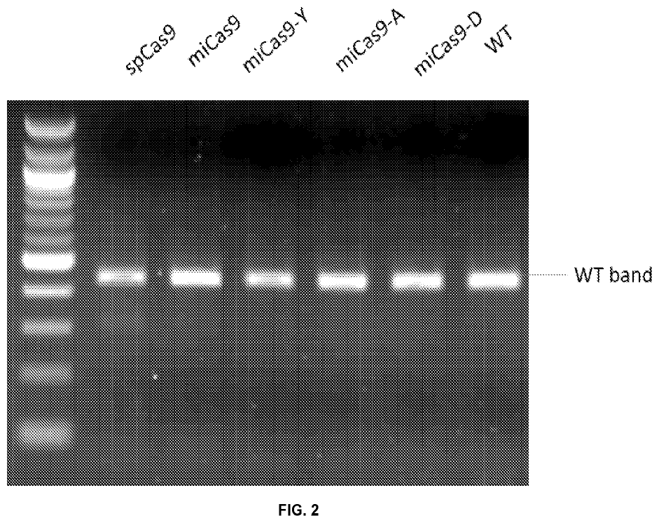

Next, during the development of embodiments of the technology provided herein, experiments were conducted to evaluate the on-target indel rates produced using the Cas9-BE27 variant fusion proteins and the sg-FANCF2 guide RNA or the sg-VEGFA3 guide RNA by T7E1 assay. Results are shown in FIG. 2. The lane marked “WT” in FIG. 2 or FIG. 3 is a positive control marker for the size of the WT band expected by the T7E1 assay.

As indicated by the data provided in FIGS. 2 and 3, the miCas9-Y, miCas9-A, and miCas9-D fusion proteins did not produce detectable indels at either the FANCF2 (FIG. 2, lanes labeled “miCas9-Y”, “miCas9-A”, and “miCas9-D”, respectively) or VEGFA3 locus (FIG. 3, lanes labeled “miCas9-Y”, “miCas9-A”, and “miCas9-D”, respectively). Indels were produced by spCas9 (FIGS. 2 and 3, lanes labeled “spCas9”) and Cas9-BE27 (FIGS. 2 and 3, lanes labeled “miCas9”) at detectable rates higher than the rates of indel production by the miCas9-Y, miCas9-A, and miCas9-D fusion proteins. These data indicate that the S15A, S22Y, and S22D substitutions in BE27, and, consequently, the miCas9-Y, miCas9-A, and miCas9-D fusion proteins significantly reduced on-target indel rates, e.g., to nonexistent, minimal, and/or undetectable levels.

### Example 4—Cas9-BE Variant Fusion Proteins Produce Large-Size Gene Knock-Ins

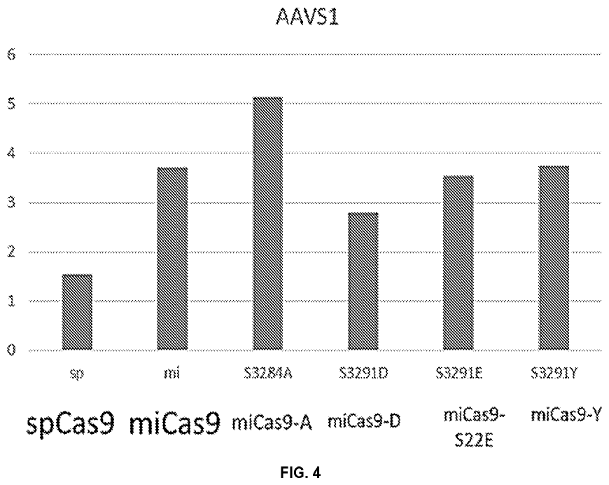

During the development of embodiments of the technology provided herein, experiments were conducted to evaluate production of large-size gene knock-ins by the miCas9-Y, miCas9-A, and miCas9-D fusion proteins. Results are shown in FIG. 4.

In FIG. 4, the percentages of GFP positive (% GFP+) cells were used as an indicator of knock-in (KI) events. The Cas9-BE27 previously described produced a greater than 2-fold increase in the % GFP+ cells (FIG. 4, bar labeled “miCas9”), which was similar to the increase provided by the miCas9-D, miCas9-S22E, and miCas9-Y variants (FIG. 4, bars labeled “miCas9-D”, “miCas9-S22E”, and “miCas9-Y”, respectively). The miCas9-A protein fusion was observed to produce a greater than 4-fold increase in % GFP+ cells (FIG. 4, bar labeled “miCas9-Y”).

These data indicate that the BE27 substitutions S15A, S22D, and S22Y do not decrease the Cas9 nuclease activity for producing double stranded breaks (DSBs), e.g., to produce knockins. In particular, the new Cas9-BE27 variant fusions (miCas9-A, miCas9-D, miCas9-E, and miCas9-Y) increase KI rates to a level that is comparable to or higher than the KI rates produced by the Cas9-BE27 fusion protein previously described and by the wild-type spCas9.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A GEN-BE27 variant fusion protein comprising a gene editing nuclease domain and a BE27 variant domain.

2. The GEN-BE27 variant fusion protein of claim 1 wherein said gene editing nuclease domain comprises a CRISPR-associated system protein, a portion thereof, a homolog thereof, or a modified version thereof.

3. (canceled)

4. The GEN-BE27 variant fusion of claim 1 wherein said gene editing nuclease domain comprises a Cas protein selected from the group consisting of Cas1, Cas1B, Cas2, Cas3, Cas4, Cas5, Cas6, Cas7, Cas8, Cas9, Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, and xCas9; a portion thereof; a homolog thereof; or a modified version thereof.

5. The GEN-BE27 variant fusion of claim 1 wherein said gene editing nuclease domain comprises a TALEN, a portion thereof, a homolog thereof, or a modified version thereof.

6. The GEN-BE27 variant fusion of claim 1 wherein said gene editing nuclease domain comprises ZFN, a portion thereof, a homolog thereof, or a modified version thereof.

7. (canceled)

8. The GEN-BE27 variant fusion of claim 1 comprising a plurality of BE27 variant domains.

9. (canceled)

10. The GEN-BE27 variant fusion of claim 1 wherein said BE27 variant domain comprises an S15A substitution, an S22Y substitution, and/or an S22D substitution.

11. (canceled)

12. (canceled)

13. A composition comprising the GEN-BE27 variant fusion protein of claim 1.

14. The composition of claim 13 further comprising a gRNA.

15. The composition of claim 13 further comprising a donor nucleic acid.

16. The composition of claim 13 further comprising a target nucleic acid.

17. (canceled)

18. (canceled)

19. The composition of claim 13 further comprising a RAD51 protein or a nucleic acid encoding a RAD51 protein.

20. (canceled)

21. The composition of claim 13 further comprising a nucleic acid comprising a knockin.

22. (canceled)

23. A method of producing a knockin in a target nucleic acid, the method comprising contacting a target nucleic acid with a GEN-BE27 variant fusion protein.

24. The method of claim 23, wherein the method comprises contacting a target nucleic acid with a ribonucleoprotein comprising the GEN-BE27 variant fusion protein and a gRNA comprising a sequence complementary to the target nucleic acid.

25. The method of claim 24, further comprising providing a donor nucleic acid comprising a knockin sequence.

26. (canceled)

27. (canceled)

28. The method of claim 24, wherein said GEN-BE27 variant fusion protein comprises a BE27 variant domain comprising an S15A substitution, an S22Y substitution, and/or an S22D substitution.

29. (canceled)

30. (canceled)

31. The method of claim 24, wherein said GEN-BE27 variant fusion protein comprises a gene editing nuclease domain that is a CRISPR-associated system protein, a portion thereof, a homolog thereof, or a modified version thereof.

32. (canceled)

33. The method of claim 24, wherein said GEN-BE27 variant fusion protein comprises a gene editing nuclease domain that is a Cas protein selected from the group consisting of Cas1, Cas1B, Cas2, Cas3, Cas4, Cas5, Cas6, Cas7, Cas8, Cas9, Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, and xCas9; a portion thereof; a homolog thereof; or a modified version thereof.

34. The method of claim 24, wherein said GEN-BE27 variant fusion protein comprises a gene editing nuclease domain that is a TALEN, a portion thereof, a homolog thereof, or a modified version thereof.

35. The method of claim 24, wherein said GEN-BE27 variant fusion protein comprises a gene editing nuclease domain that is a ZFN, a portion thereof, a homolog thereof, or a modified version thereof.

36-47. (canceled)


SEQUENCE LISTING (20 sequences)

SEQ ID NO: 1 - protein, 36 aa, Homo sapiens
ALDFLSRLPLPPPVSPICTFVSPAAQKAFQPPRSCG

SEQ ID NO: 2 - DNA, 23 bp, Artificial sequence
  Synthetic
gagttagagcagaagaagaaagg

SEQ ID NO: 3 - DNA, 23 bp, Artificial sequence
  Synthetic
ggtgagtgagtgtgtgcgtgtgg

SEQ ID NO: 4 - DNA, 23 bp, Artificial sequence
  Synthetic
gctgcagaagggattccatgagg

SEQ ID NO: 5 - DNA, 23 bp, Artificial sequence
  Synthetic
ggggccactagggacaggattgg

SEQ ID NO: 6 - RNA, 34 nt, Artificial sequence
  Synthetic
  misc_feature (1)..(12): n is a, c, g, or u
nnnnnnnnnnnnguuuaagagcuaugcuguuuug

SEQ ID NO: 7 - protein, 108 aa, Artificial sequence
  Synthetic
GCNYTNGAYTTYYTNWSNMGNYTNCCNYTNCCNCCNCCNGTNWSNCCNATHTGYACNTTYGTNWSNCCNGCNGCNCARAARGCNTTYCARCCNCCNMGNWSNTGYGGN

SEQ ID NO: 8 - DNA, 108 bp, Homo sapiens
gccttggatttcttgagtagactgcctttacctccacctgttagtcccatttgtacatttgtttctccggctgcacagaaggcatttcagccaccaaggagttgtggc

SEQ ID NO: 9 - protein, 1368 aa, Streptococcus pyogenes Cas9 [sequence omitted]

SEQ ID NO: 10 - protein, 36 aa, Streptococcus pyogenes Cas9
ALDFLSRLPLPPPVAPICTFVSPAAQKAFQPPRSCG

SEQ ID NO: 11 - protein, 36 aa, Streptococcus pyogenes Cas9
ALDFLSRLPLPPPVSPICTFVYPAAQKAFQPPRSCG

SEQ ID NO: 12 - protein, 36 aa, Streptococcus pyogenes Cas9
ALDFLSRLPLPPPVSPICTFVDPAAQKAFQPPRSCG

SEQ ID NO: 13 - DNA, 20 bp, Artificial sequence
  Synthetic
atgtggctctggttctgggt

SEQ ID NO: 14 - DNA, 20 bp, Artificial sequence
  Synthetic
ggaaggaggaggcctaagga

SEQ ID NO: 15 - DNA, 20 bp, Artificial sequence
  Synthetic
tcccaggtgctgacgtaggt

SEQ ID NO: 16 - DNA, 20 bp, Artificial sequence
  Synthetic
gagagtcgccgtctccaagg

SEQ ID NO: 17 - DNA, 20 bp, Artificial sequence
  Synthetic
agcccattccctctttagcc

SEQ ID NO: 18 - DNA, 18 bp, Artificial sequence
  Synthetic
ggagtgacccctggcctt

SEQ ID NO: 19 - DNA, 25 bp, Artificial sequence
  Synthetic
gtggggagatttgcatctgtggagg

SEQ ID NO: 20 - DNA, 26 bp, Artificial sequence
  Synthetic
gcttttataccatcttggggttacag